(19) United States
(12) Patent Application Publication
(10) Pub. No.: US 2010/0163068 A1
(43) Pub. Date: Jul. 1, 2010

(54) NAIL VARNISH COMPRISING AT LEAST ONE POLYSACCHARIDE ESTER OR ALKYL ETHER AND AT LEAST ONE PLANT RESIN

(75) Inventors: Guillaume KERGOSIEN, Chaville (FR); Frederic LEURIDAN, Brie Comte Robert (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 12/638,698

(22) Filed: Dec. 15, 2009

Related U.S. Application Data
(60) Provisional application No. 61/153,322, filed on Feb. 18, 2009.
(60) Provisional application No. 61/153,328, filed on Feb. 18, 2009.
(60) Provisional application No. 61/202,024, filed on Jan. 21, 2009.

(30) Foreign Application Priority Data
Dec. 17, 2008 (FR) 08 58695
Dec. 17, 2008 (FR) 08 58696
Dec. 23, 2008 (FR) 08 59040

(51) Int. Cl. A45D 29/00; A61K 8/73; A61K 8/97; A61K 8/02

(52) U.S. Cl. 132/200; 132/73; 424/61; 424/401

(57) ABSTRACT

A subject-matter of the present invention is a nail varnish comprising a solvent medium composed of one or more solvents of plant origin, at least one polysaccharide ester or alkyl ether and at least one plant resin.

An additional subject-matter of the invention is a cosmetic method for making up or for the non-therapeutic care of the nails comprising the application, to the nails, of at least one layer of a nail varnish in accordance with the invention.

Finally, the invention relates to a flexible article comprising, in particular in an adhesive layer, at least one plant resin, at least one plasticizer and preferably at least one solvent of plant origin, characterized in that the plasticizer is chosen from caprylic and/or capric acid esters or an ester of caprylyl and/or capryl alcohol, and especially caprylic and/or capric acid triglycerides.

A subject-matter of the present invention is a nail varnish composition comprising a solvent medium composed of one or more solvents of plant origin, at least one polysaccharide ester or alkyl ether and at least one plant resin.

This composition can be applied to human nails or else to false nails.

The present invention additionally relates to a corresponding method for making up and/or for the non-therapeutic care of the nails.

The coloured or transparent nail varnish composition can be employed as varnish base or base coat, as product for making up the nails, as finishing composition, also known as top coat, to be applied to the product for making up the nails, or else as product for the cosmetic care of the nails.

In this day and age, it is preferable, for reasons of allergy to the varnishes, to avoid the use of certain compounds and to replace them by other less harmful compounds, such as, for example, compounds of plant origin.

Thus, the document WO 2006/056558 describes nail varnish compositions comprising a solvent exclusively of plant origin based in particular on fusel oil derivatives.

Likewise, the document EP 0 676 451 is targeted at aqueous dispersions of plant resins (shellac resin, gum sandarac, elemis, dammars and copal) in order to replace synthetic resin pseudolatexes.

Furthermore, Application WO 2007/080172 provided a plasticizer of carbonate type of plant origin in a nail varnish composition.

Likewise, the document U.S. Pat. No. 5 066 484 describes nail varnishes comprising a plasticizer of triglycerides type.

A description is also given, in U.S. Pat. No. 5 145 670, of nail varnish compositions comprising di- or triesters as plasticizing agent and exhibiting improved cosmetic properties.

Likewise, the document JP 2005 263656 teaches a dental varnish composition comprising a plant resin (benzoin gum).

Furthermore, the document JP 2004 315479 is targeted at nail varnish compositions comprising a caprylic acid monoglyceride as plasticizing agent.

It is commonplace to use, in nail varnish compositions, film-forming agents in order to obtain, after deposition on the nail, a resistant film which exhibits good wear. Currently, nitrocellulose still remains the most commonly used film-forming agent in nail varnishes exhibiting optimized wear properties.

However, these nail varnishes comprising nitrocellulose exhibit the disadvantage of not being stable towards heat and on storage.

This results in particular in yellowing of these compositions over time.

Furthermore, the introduction of large amounts of nitrocellulose conditions the composition of the organic solvent phase of the nail varnish, which then has to be compatible with this film-forming agent.

Furthermore, it is useful to remember that the formulation of the nail varnishes is problematic as the composition requires a judicious adjustment between the various constituents. Changing one of these constituents can greatly modify the cosmetic properties of the varnish and render it unsuitable for use.

It is a specific aim of the present invention to overcome the above disadvantages and to provide a nail varnish composition comprising a reduced content of nitrocellulose, indeed even devoid of nitrocellulose, exhibiting a good stability over time and making possible the formation of a homogeneous, glossy and/or nonbrittle film.

The inventors have thus discovered that it is possible to obtain such a composition comprising less than 5% of nitrocellulose dry matter with respect to the total weight of the composition, indeed even devoid of nitrocellulose, by using a solvent medium composed of one or more solvents of plant origin, at least one polysaccharide ester or alkyl ether and at least one plant resin.

Thus, a first aspect of the invention relates to a nail varnish comprising a solvent medium composed of one or more solvents of plant origin, at least one polysaccharide ester or alkyl ether and at least one plant resin, characterized in that the solvent medium is chosen from a mixture of ethanol and ethyl acetate.

According to another aspect, the invention relates to a nail varnish comprising a solvent medium composed of one or more solvents of plant origin, at least one polysaccharide ester or alkyl ether and at least one plant resin, characterized in that the plant resin is chosen from rosin and its derivatives, terpene resins, gum sandarac, dammars, elemi, copals, benzoin, mastic gum and their mixtures.

Such a nail varnish according to these aspects makes possible in particular good stability over time and makes it possible in particular to obtain a solid, indeed even hard, glossy, adherent and/or nonbrittle film.

According to yet another aspect, a subject-matter of the invention is a cosmetic method for making up or for the non-therapeutic care of the nails comprising the application, to the nails, of at least one layer of a nail varnish as defined above.

According to yet another of its aspects, a subject-matter of the invention is the use of a nail varnish as defined above for obtaining, after deposition on the nail, a homogenous, glossy and nonbrittle film.

The nail varnish according to the invention comprises a cosmetically acceptable medium, that is to say a nontoxic medium capable of being applied to human keratinous substances, in particular the nails.

The present invention also relates to a made-up synthetic support comprising a make-up obtained by the method of the invention.

According to yet another aspect, the present invention relates to a nail varnish comprising at least one caprylic/capric alcohol and/or acid ester, at least one polysaccharide derivative which has not been subjected to reaction with alkylene oxides especially a polysaccharide alkyl ether or ester and at least one coresin, especially a plant resin, the caprylic/capric alcohol and/or acid ester corresponding to one of the following formulae (I) to (X):

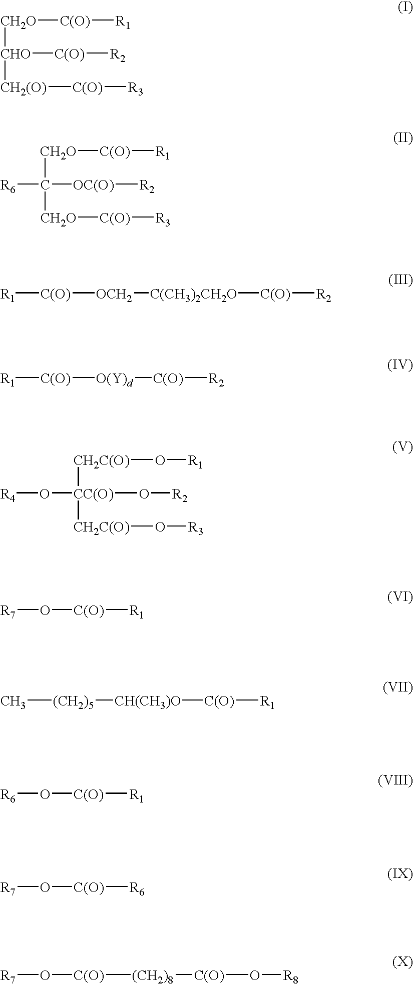

in which:

R₁, R₂ and R₃, which are identical or different, represent the hydrogen atom or a saturated, linear or branched, preferably linear, alkyl chain comprising 7 or 9 carbon atoms,

R₄ represents the hydrogen atom or an (Alk)-C(CO)— group in which (Alk) represents a linear or branched alkyl chain comprising between 1 and 5 carbon atoms,

R₆ represents a substituted or unsubstituted and linear or branched alkyl chain comprising between 1 and 18 carbon atoms, in particular between 1 and 10 carbon atoms, or a substituted or unsubstituted and linear or branched alkenyl chain comprising between 2 and 18 carbon atoms, in particular between 2 and 10 carbon atoms,

Y is an ethoxy, isopropoxy or propoxy group,

d is between 1 and 1000, in particular between 1 and 15, in particular equal to 1, and

R₇ and R₈, which are identical or different, represent a saturated, linear or branched, preferably linear, alkyl chain comprising 8 or 10 carbon atoms.

According to a particular embodiment, the caprylic/capric alcohol and or acid ester is a caprylic and/or capric acid triglyceride.

According to this aspect, the nail varnish exhibits excellent cosmetic properties, among which may be mentioned the absence of irritation of the nails, good application, the production of a homogeneous film having excellent gloss and a rapid drying time for the film but also good adhesion to the surface of the support, a degree of flexibility and good resistance of the film, for example for the purpose of preventing cracks and the flaking thereof, and makes possible the reduction in, indeed even the removal of, the normal plasticizers.

A nail varnish according to this aspect makes possible in particular good stability over time and makes it possible to obtain a solid, glossy and/or nonbrittle film.

According to a specific embodiment, the coresin is a plant resin as defined below.

According to yet another specific embodiment, the polysaccharide derivative which has not been subjected to reaction with alkylene oxides is a polysaccharide ester or alkyl ether as defined below.

According to yet another of its aspects, the invention relates to a nail varnish comprising (i) at least one caprylic/capric alcohol and/or acid ester, in particular corresponding to one of the formulae (I):

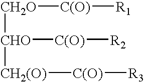

where R₁, R₂ and R₃ are as defined above,

or (II) to (X) as defined above, (ii) at least one polysaccharide derivative which has not been subjected to reaction with alkylene oxides especially a polysaccharide alkyl ether or ester and (iii) at least one coresin, especially a plant resin, and comprising less than 5% of nitrocellulose dry matter, with respect to the total weight of the nail varnish, indeed even being devoid of nitrocellulose.

According to one of its aspects, the nail varnish exhibits a liquid or gelled texture; it differs in particular from a patch or a soft article comprising at least one layer of polymer and one layer of adhesive intended to be adhesively bonded and adjusted by virtue of its properties of being able to change in shape on the nail.

In other words, the nail varnish according to the invention is typically applied in the form of superimposed layers at the surface of the nails or false nails to be made up, for example using a brush.

The nail varnish according to the invention may be in various forms: either in a standard nail varnish form, i.e. having a liquid or gelled texture as outlined above, or in the form of an article that is placed on and adjusts to the nails, as outlined hereinbelow,

The present invention is specifically directed towards proposing a method for making up or caring for the nails or false nails with bonding power, the composition of which allows sufficient flexibility and stretchability at the time of application.

The flexible article according to the invention comprises, in particular in an adhesive layer, at least one plant resin, at least one plasticizer and preferably at least one solvent of plant origin, characterized in that the plasticizer is chosen from caprylic and/or capric acid esters or an ester of caprylyl and/or capryl alcohol and especially caprylic and/or capric acid triglycerides.

The flexible article may especially comprise a polymer layer and an adhesive layer.

According to one particular embodiment, the plant resin, the plasticizer and the possible solvent of plant origin are included in the adhesive layer.

In one variant, the article may comprise a superposition of polymer layers.

In another variant, the article may comprise one or more adhesive layers in addition to the polymer layer(s).

According to another aspect, the present invention relates to a product for making up and/or caring for the nails and/or false nails, comprising, in substantially airtight packaging, at least one article in accordance with the invention, the packaging being such that the article therein is kept in a partially dry form.

This flexible article is referred to in the rest of the description, for the sake of simplicity, as a “partially dry flexible article”.

According to this aspect of the invention, the partially dry flexible article does not acquire a totally dry appearance, and thus its definitive form, until after it has been applied to the nail and undergone drying, by simple exposure to the ambient air.

According to another aspect, the present invention relates to a process for making up and/or caring for the nails and/or false nails, comprising at least one step that consists in applying to a natural nail and/or a false nail a flexible article according to the invention, whether it is considered in dry form or in partially dry form.

According to one embodiment, the flexible article in accordance with the present invention may be in various forms, such as a star, a square, a circle, etc.

For the purposes of the present invention, the term “partially dry” is intended to qualify the fact that the article obtained after forming the film is not entirely free of residual solvent. In particular, it has a solids content of greater than 80% and more particularly greater than 85% and less than 95% by weight relative to its total weight.

In contrast, the flexible article according to the invention, which is the subject matter of the first aspect of the invention mentioned above, is referred to as a “flexible article in dry form”.

I—Nail Varnish Composition in Liquid or Gelled Form

Solvent of Plant Origin

According to a specific embodiment, a nail varnish according to the invention can comprise a solvent medium composed of one or more solvents of plant origin.

In other words, according to this embodiment, the composition is then completely devoid of solvents of non-plant origin.

Within the meaning of the invention, “solvent of plant origin” is understood to mean a solvent which is obtained directly from plants and which has not been subjected to any chemical modification after extraction.

The solvents of plant origin in accordance with the invention are preferably chosen from alcohols and/or acetates and/or lactates.

According to a preferred embodiment, the solvent of plant origin is chosen in particular from ethanol and/or ethyl acetate and/or ethyl lactate.

Preferably, it will be a mixture of ethanol and ethyl acetate.

Ethanol exclusively of plant origin is widely available from numerous suppliers, in particular from Tereos. Ethyl acetate of plant origin is available from Rhodia. Ethyl lactate is available from React-NTI under the reference Envirosolve Silver.

According to a specific embodiment, the cosmetic compositions according to the invention comprise from 1 to 99% of solvent medium by weight, in particular from 40 to 90% by weight, especially from 60 to 80% by weight, with respect to the total weight of the composition.

Polysaccharide Ester or Alkyl Ether

According to one embodiment, the nail varnish composition according to the invention comprises a polysaccharide ester or alkyl ether.

“Polysaccharide ester or alkyl ether” denotes, within the meaning of the invention, a polysaccharide formed of repeat units comprising at least two identical or different rings and exhibiting a degree of substitution per saccharide unit of between 1.9 and 3, preferably of between 2.2 and 2.9 and more particularly of between 2.4 and 2.8. Substitution denotes the functionalization of the hydroxyl groups to give ester and/or alkyl ether functional groups and/or the functionalization of the carboxyl groups to give ester functional groups.

In other words, the invention denotes a polysaccharide partially or completely substituted by ester and/or alkyl ether groups.

The hydroxyl groups can preferably be substituted by ester functional groups and/or alkyl ether functional groups comprising from 2 to 4 carbon atoms.

The saccharide units can in particular be chosen from mannose, galactose, glucose, furanose, rhamnose, arabinose, xylose, fucose, glucuronic acid, mannuronic acid and galacturonic acid.

Mention may made, among appropriate compounds, of cellulose esters and/or alkyl ethers, the unit of which is composed of glucose, guar gum or locust bean gum esters and/or alkyl ethers, the units of which are composed of galactose and mannose, konjac esters and/or alkyl ethers, the unit of which is composed of glucose and mannose, and tamarind esters and/or alkyl ethers, the unit of which is composed of galactose, xylose and glucose.

The choice will in particular be preferably made of cellulose acetate butyrates, cellulose acetate propionates, ethyl celluloses and ethyl guars.

Mention may in particular be made, as polysaccharide ester or alkyl ether, of those sold under the trade names Aqualon by Hercules and Ethocel by Dow and under the names CAP and CAB by Eastman.

More particularly, the polysaccharide ester or alkyl ether is chosen from ethyl guars, ethyl celluloses, cellulose acetate propionates or cellulose acetate butyrates.

The cosmetic composition according to the present invention can comprise at least one and in particular at least two polysaccharide esters and/or alkyl ethers.

According to a specific embodiment, the nail varnish according to the invention comprises at least 1% by weight of polysaccharide ester or alkyl ether, preferably at least 2% by weight, indeed even at least 3% by weight, with respect to the total weight of the nail varnish.

According to a specific embodiment, the nail varnish according to the invention comprises from 1 to 60% by weight, in particular from 2 to 30% by weight, especially from 4 to 10% by weight, of polysaccharide ester or alkyl ether, with respect to the total weight of the nail varnish.

Plant Resin

According to one embodiment, a nail varnish in accordance with the invention comprises at least one plant resin.

The plant resins in accordance with the invention are chosen from:

rosin or its derivatives, terpene resins, and

gum sandarac, dammars, elemi, copals, benzoin or mastic gum.

Rosin is a mixture predominantly comprising organic acids known as rosin acids (mainly acids of abietic type and of pimaric type).

Three types of rosin exist: the rosin (gum rosin) obtained by incision on living trees, wood rosin, which is extracted from pine stumps or wood, and tall oil (tall oil rosin), which is obtained from a byproduct originating from the production of paper.

The rosin derivatives can result in particular from the polymerization, hydrogenation and/or esterification (for example with polyhydric alcohols, such as ethylene glycol, glycerol or pentaerythritol) of rosin acids. Mention may be made, for example, of the rosin esters sold under the references Foral 85, Pentalyn H and Staybelite Ester 10 by Hercules, Sylvatac 95 and Zonester 85 by Arizona Chemical or Unirez 3013 by Union Camp.

The terpene resins typically result from the polymerization of at least one monomer chosen from α-pinene, β-pinene, limonene and their mixtures. These resins can exhibit a molecular weight ranging from 300 to 2000 g/mol. Such resins are sold, for example, under the names Piccolyte A115 and 5125 by Hercules or Zonarez 7100 or Zonatac 105 Lite by Arizona Chem.

The cosmetic composition according to the present invention can comprise at one and in particular at least two plant resins.

According to a specific preferred embodiment, the plant resins are more particularly gum sandarac, benzoin or elemi.

The nail varnish according to the invention comprises from 1 to 60% by weight, in particular from 10 to 50% by weight, especially from 15 to 35% by weight, of plant resin, with respect to the total weight of the composition.

Advantageously, the nail varnish comprises less than 5% by weight of water, indeed even less than 3% by weight of water, indeed even less than 1% by weight of water, with respect to the total weight of the nail varnish, indeed even is completely devoid thereof.

According to one embodiment of the invention, a nail varnish composition according to the invention comprises less than 5% by weight of nitrocellulose dry matter, with respect to the total weight of the composition, preferably less than 3% by weight, preferably less than 1.5% by weight, preferably less than 1% by weight.

According to another embodiment of the invention, the nail varnish composition according to the invention is completely devoid of nitrocellulose.

According to one embodiment of the invention, a nail varnish comprises at least one polysaccharide ester or alkyl ether, in particular ethyl guar or cellulose acetate butyrate, at least one plant resin, in particular benzoin gum, elemi gum or sandarac resin, and at least one solvent medium composed of a mixture of ethanol and ethyl acetate,

Polysaccharide Derivative Which Has Not Been Subjected to Reaction with Alkylene Oxides

According to another embodiment of the invention, the nail varnish composition comprises a polysaccharide derivative which has not been subjected to reaction with alkylene oxides.

“Polysaccharide derivative which has not been subjected to reaction with alkylene oxides” denotes, within the meaning of the invention, a polysaccharide derivative formed of repeat units comprising at least two identical or different rings which has not been subjected to reaction with alkylene oxides, such as ethylene oxide or propylene oxide.

Mention is made, as polysaccharide derivative which has not been subjected to reaction with alkylene oxides, in addition to the polysaccharide esters and alkyl ethers described above, of nitrocellulose.

Mention may in particular be made, as polysaccharide derivative which has not been subjected to reaction with alkylene oxides, of that sold under the trade name Nitrocellulose by Bergerac.

The cosmetic composition according to the present invention can comprise at least one and in particular at least two polysaccharide derivatives which have not been subjected to reaction with alkylene oxides in accordance with the invention.

According to a specific embodiment, the nail varnish according to the invention comprises from 1 to 60% by weight, in particular from 2 to 30% by weight, especially from 4 to 10% by weight, of polysaccharide derivative which has not been subjected to reaction with alkylene oxides, with respect to the total weight of the nail varnish.

Coresin

According to a specific embodiment, the nail varnish composition according to the invention comprises a coresin.

This coresin can preferably be, in addition to the plant resins described above, a fatty acid ester, such as the alkyd resins described below, or also be a resin of plant origin chosen from:

gum arabic, guar gum, xanthan derivatives or karaya gum;

alginates, carrageenans, ulvans and other algal colloids;

caprolactams, pullulans, pectins, mannan, galactomannans, glucomannans and their derivatives.

The alkyd resins contribute to the formation of a film on the nail and make it possible to improve the gloss and the adhesion of the film.

The alkyd resins used according to the invention are polyesters comprising hydrocarbon chains of fatty acids. Such resins are described in particular in the Kirk-Othmer Encyclopedia of Chemical Technology, 4th edition, volume 2, pages 53 to 63, the content of which is incorporated by way of reference in the present patent application. These resins are obtained by polymerization of polyols and polyacids or their corresponding anhydride in the presence of fatty acids. These fatty acids can be employed as is or else in the form of fatty acid triglycerides or alternatively can be present in the oils during the synthesis of the alkyd resin.

Due to the presence of hydrocarbon chains of fatty acids in the alkyd resin, alkyd resins are commonly defined by their oil length. Thus, according to the present patent application, oil length of an alkyd resin is understood to mean the percentage by weight of hydrocarbon chains of fatty acids present in the alkyd resin.

Mention may be made, as polyols which can be employed for the synthesis of alkyd resins, of pentaerythritol, trimethylolpropane, trimethylolethane, neopentyl glycol, propylene glycol, ethylene glycol, 1,6-hexanediol, 1,4-butanediol, diethylene glycol and, in particular, glycerol.

Mention may in particular be made, as polyacid or anhydride, of isophthalic acid, terephthalic acid, trimellitic anhydride, maleic anhydride, adipic acid, fumaric acid, azelaic acid, sebacic acid and, in particular, phthalic anhydride.

The fatty acids used to prepare the alkyd resins present in the compositions according to the present invention correspond to the formula R—COOH, in which R denotes a saturated or unsaturated hydrocarbon radical having from 7 to 45 carbon atoms approximately, preferably from 9 to 35 carbon atoms, advantageously from 15 to 35 carbon atoms and better still from 15 to 21 carbon atoms.

Mention may in particular be made, as fatty acids, of palmitic acid, stearic acid, oleic acid, ricinoleic acid, linoleic acid, linolenic acid and, in particular, capric acid.

Fatty acids are present in the majority of oils of natural origin, in particular in the form of triglycerides. The triglycerides of fatty acids are esters resulting from the reaction of the three alcohol functional groups of glycerol with fatty acids, it being possible for these fatty acids to be identical or different. Oils of natural origin can thus be used during the polymerization. They can be chosen from linseed oil, China wood oil, oiticica oil, soybean oil, sunflower oil, safflower oil, castor oil, coconut oil, olive oil, palm oil, rapeseed oil, peanut oil and tall oil.

Mention may in particular be made, as alkyd resins which can be used according to the invention, of those sold under the names “Beckosol ODE 230 70E” by Dainippon Ink & Chem (phthalic anhydride/glycerol/glycidyl decanoate copolymer in ethyl acetate at 70%), “Necowel 581®” (50% in soybean oil), “Necowel 585®” (20% in sunflower oil), “Necowel 580®” (20% in sunflower oil), “Necowel 586 N®” (50% in soybean oil), “Necowel EP 1161®” (50% in soybean oil), “Necowel EP 1213®” (20% in oil), “Necowel EP 2009®” (32% in sunflower oil), “Necowel EP 2019®” (20% in oil), “Necowel EP 2275®” (35% in oil), “Necowel EP 2329®” (34% in oil), or “Necowel EP 3016®” (30% in oil) by Ashland or “Uradil XP 515 AZ®” (73% in tall oil) or “Uradil XP 516 AZ®” (63% in tall oil) by DSM Resins.

According to a preferred embodiment, the coresin is Beckosol ODE 230 70E (phthalic anhydride/glycerol/glycidyl decanoate copolymer in ethyl acetate at 70%).

The coresin present in the composition according to the invention can take the form of a mixture of several resins.

According to a specific embodiment, the nail varnishes according to the invention comprise from 1 to 60% by weight, in particular from 2 to 40% by weight, especially from 4 to 30% by weight, of coresin, with respect to the total weight of the nail varnish.

Caprylic and/or Capric Alcohol and/or Acid Ester

According to one embodiment, a nail varnish according to the invention can additionally comprise a caprylic and/or capric alcohol and/or acid ester, or, in other words, an ester of caprylic and/or capric acid, an ester of caprylic and/or capric alcohol, or their mixture, as plasticizer, the said ester being of plant origin. Caprylic and/or capric alcohol or acid is understood to mean linear or branched, preferably linear, alcohols or carboxylic acids having 8 or 10 carbons.

Within the meaning of the present invention, caprylic and/or capric is synonymous with caprylic/capric.

Within the meaning of the invention, “ester of plant origin” is understood to mean an ester which is obtained from plant starting materials or starting materials of plant origin.

This ester can correspond to one of the following formulae (I) to (X):

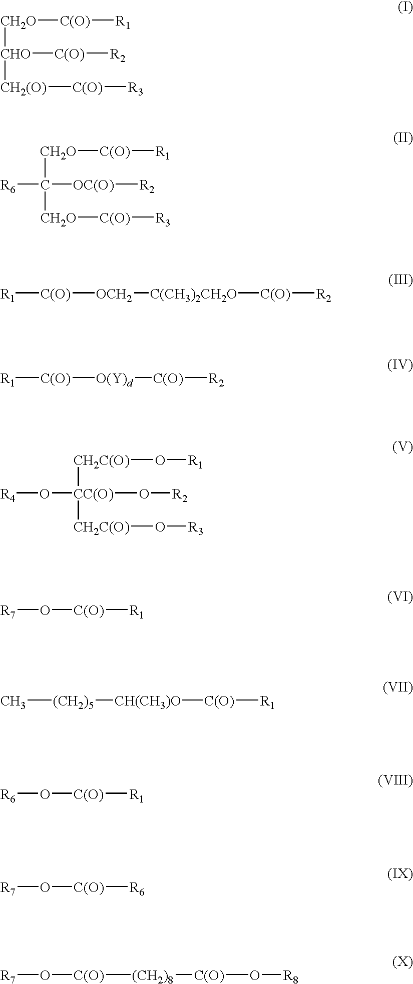

in which:

R₁, R₂ and R₃, which are identical or different, represent the hydrogen atom or a saturated, linear or branched, preferably linear, alkyl chain comprising 7 or 9 carbon atoms,

R₄ represents the hydrogen atom or an (Alk)-C(CO)— group in which (Alk) represents a linear or branched alkyl chain comprising between 1 and 5 carbon atoms,

R₆ represents a substituted or unsubstituted and linear or branched alkyl chain comprising between 1 and 18 carbon atoms, in particular between 1 and 10 carbon atoms, or a substituted or unsubstituted and linear or branched alkenyl chain comprising between 2 and 18 carbon atoms, in particular between 2 and 10 carbon atoms,

Y is an ethoxy, isopropoxy or propoxy group,

d is between 1 and 1000, in particular between 1 and 15, in particular equal to 1, and

R₇ and R₈, which are identical or different, represent a saturated, linear or branched, preferably linear, alkyl chain comprising 8 or 10 carbon atoms.

The ester in accordance with the invention can also comprise a mixture of different esters defined above.

Such esters are commercially available, for example from Cognis or RTD Hallstar.

Preferably, such esters are chosen from the caprylic/capric acid esters as mentioned above. Preferably, such esters are chosen from the compounds of formulae (I), (VI), (VII) and (VIII). Preferably, such esters are chosen from caprylic/capric acid esters which are linear.

According to a preferred embodiment of the invention, this plasticizer can in particular be a compound of formula (I) as defined above or, in other words, a caprylic and/or capric acid triglyceride.

According to another preferred embodiment of the invention, this plasticizer can in particular be a compound of formula (VII) as defined above or, in other words, an isooctyl caprylate and/or caprate.

According to a specific embodiment, the cosmetic compositions according to the invention comprise from 0.5 to 15% by weight, in particular from 1 to 10% by weight, especially from 2 to 8% by weight, of caprylic and/or capric alcohol and/or acid ester, with respect to the total weight of the composition.

Liquid Fatty Phase

The nail varnishes in accordance with the present invention can additionally comprise a liquid fatty phase, preferably of natural origin. Oils of natural origin, whether or not they have been subjected to conversion operations, such as transesterification or hydrogenation or a fractionation, are thus suitable for the invention.

Oil is understood to mean any fatty substance in the liquid form at ambient temperature (20-25° C.) and at atmospheric pressure. The liquid fatty phase can also comprise, in addition to the oils, other compounds dissolved in the oils, such as gelling and/or structuring agents.

Mention may be made, as examples of oils suitable for the implementation of the present invention, of glycols of natural origin, such as glycerol or propylene glycol, the glycol ethers of natural origin, and oils of natural origin chosen from oils comprising at least one fatty acid chosen from caprylic acid, capric acid, lauric acid, myristic acid, palmitic acid, stearic acid, oleic acid, ricinoleic acid, linoleic acid, linolenic acid, arachidic acid, gadoleic acid, behenic acid, erucic acid, brassidic acid, cetoleic acid, lignoceric acid or nervonic acid, and a mixture of these.

The nail varnish according to the present invention can comprise at least one and in particular at least two oils.

The oils can be hydrocarbon oils of plant origin.

Within the meaning of the present invention, “hydrocarbon oil” is understood to mean an oil comprising mainly hydrogen and carbon atoms and optionally oxygen, nitrogen, sulphur and/or phosphorous atoms.

According to a specific embodiment, the cosmetic compositions in accordance with the invention can comprise at least one oil chosen from the following vegetable hydrocarbon oils: the isopropyl myristate sold by Stearinerie Dubois; the triglycerides of caprylic/capric acid which are sold by Stepan; the hybrid rapeseed oil, the liquid cottonseed oil, the refined protected deodorized mango oil, the liquid fraction of protected shea butter and the refined canola seed oil which are sold by Karlshamns; the Lipex Sheasoft and the cottonseed oil which are sold by Karlshamns; the deodorized apricot kernel oil sold by Nestlé; the sweet almond oil sold by Soetenaey; the peach kernel oil sold by Aarhus United; the rapeseed oil, the maize germ oil, the olive oil, the grape seed oil, the soybean oil and the sunflower oil which are sold by Huileries de Lapalisse and the walnut oil sold by Soetenaey.

In addition to the abovementioned oils, the nail varnishes in accordance with the present invention can, of course, comprise at least one other liquid fatty substance, with the proviso that the latter is present in amounts in accordance with the requirements according to the invention.

Mention may in particular be made, as hydrocarbon oil, of:

synthetic ethers having from 10 to 40 carbon atoms;

linear or branched hydrocarbons of mineral or synthetic origin, such as liquid petrolatum, polydecenes, hydrogenated polyisobutene, such as Parleam, squalane and their mixtures, and in particular hydrogenated polyisobutene, and

synthetic esters, such as oils of formula R₁COOR₂ in which R₁ represents the residue of a linear or branched fatty acid comprising from 1 to 40 carbon atoms and R₂ represents a hydrocarbon chain, in particular a branched hydrocarbon chain, comprising from 1 to 40 carbon atoms, provided that R₁+R₂≧10.

The esters can in particular be chosen from esters, in particular fatty acid esters, such as, for example:

cetearyl octanoate, esters of isopropyl alcohol, such as isopropyl myristate or isopropyl palmitate, ethyl palmitate, 2-ethylhexyl palmitate, isopropyl stearate or isostearate, isostearyl isostearate, octyl stearate, hydroxylated esters, such as isostearyl lactate or octyl hydroxystearate, diisopropyl adipate, heptanoates and in particular isostearyl heptanoate, octanoates, decanoates or ricinoleates of alcohols or of polyalcohols, such as propylene glycol dioctanoate, cetyl octanoate, tridecyl octanoate, 2-ethylhexyl palmitate and 4-diheptanoate, alkyl benzoate, polyethylene glycol diheptanoate, propylene glycol di(2-ethylhexanoate) and their mixtures, C₁₂ to C₁₅ alkyl benzoates, hexyl laurate, esters of neopentanoic acid, such as isodecyl neopentanoate, isotridecyl neopentanoate, isostearyl neopentanoate or octyldodecyl neopentanoate, esters of isononanoic acid, such as isononyl isononanoate, isotridecyl isononanoate or octyl isononanoate, or hydroxylated esters, such as isostearyl lactate or diisostearyl malate;

esters of polyols and esters of pentaerythritol, such as dipentaerythritol tetrahydroxystearate/tetraisostearate;

esters of dimer diols and dimer diacids, such as Lusplan DD-DA5® and Lusplan DD-DA7®, sold by Nippon Fine Chemical and described in Application FR 03-02809, filed on 6 Mar. 2003;

fatty alcohols which are liquid at ambient temperature with a branched and/or unsaturated carbon chain having from 12 to 26 carbon atoms, such as 2-octyldodecanol, isostearyl alcohol, oleyl alcohol, 2-hexyldecanol, 2-butyloctanol and 2-undecylpentadecanol; and dialkyl carbonates, it being possible for the 2 alkyl chains to be identical or different, such as dicaprylyl carbonate, sold under the name Cetiol CC® by Cognis.

According to a specific embodiment, the nail varnish according to the invention comprises from 0.1 to 20% by weight, in particular from 1 to 10% by weight, especially from 1 to 5% by weight, of liquid fatty phase, with respect the total weight of the nail varnish.

Additional Film-Forming Polymer

The nail varnish according to the invention can comprise, in addition to the polysaccharide ester or alkyl ether, an “additional” film-forming polymer.

“Film-forming polymer” is understood to mean, within the meaning of the present invention, a polymer capable of forming, by itself alone or in the presence of an additional agent which is able to form a film, an isolable film, in particular a continuous and adherent film, on a support, in particular on the nails.

Use may be made, in the nail varnish, of just one additional film-forming polymer or of several additional film-forming polymers.

This additional film-forming polymer can be chosen from the group consisting of synthetic polymers of radical type or of polycondensate type, polymers of natural origin and their blends,

The additional film-forming polymer can be chosen in particular from polyurethanes, acrylic polymers, vinyl polymers, polyvinyl butyrals, alkyd resins, ketone/aldehyde resins, resins resulting from aldehyde condensation products, such as arylsulphonamide/formaldehyde resins, for example toluenesulphonamide/formaldehyde resin, arylsulphonamide/epoxy resins, ethyl tosylamide resins or film-forming silicone polymers.

Use may in particular be made, as additional film-forming polymers, of the toluenesulphonamide/formaldehyde resins “Ketjentflex MS80” from Akzo or “Santolite MHP” or “Santolite MS 80” from Faconnier or “Resimpol 80” from Pan Americana, the alkyd resin “Beckosol ODE 230-70-E” from Dainippon, the acrylic resin “Acryloid B66” from Röhm & Haas, the polyurethane resin “Trixene PR 4127” from Baxenden or the acetophenone/formaldehyde resin sold under the reference synthetic resin SK by Degussa.

The additional film-forming polymer can be present in the nail varnish composition according to the invention in an amount ranging from 0.1% to 30% by weight, with respect to the total weight of the composition, preferably ranging from 0.5% to 20% by weight and better still from 1% to 10% by weight.

Gelling Agent

The nail varnish composition according to the invention can additionally comprise a gelling agent.

This gelling agent can in particular be chosen from: hydrophobic silicas, such as those described in the document EP-A-0 898 960, for example sold under the references “Aerosil R812®” by Degussa, “Cab-O-Sil TS-530®”, “Cab-O-Sil TS-610®” and “Cab-O-Sil TS-720®” by Cabot and “Aerosil R972®” and “Aerosil R974®” by Degussa, clays, such as montmorillonite, or modified clays, such as bentones, for example sold under the reference Bentone 27 V (Elementis), stearalkonium hectorite or stearalkonium bentonite.

The total proportion of gelling agent(s) in the compositions according to the invention can range from 0.01 to 15% by weight, with respect to the total weight of the composition, preferably from 0.5 to 15% by weight and better still from 0.5 to 10% by weight.

Colouring Material

The nail varnish composition according to the invention can additionally comprise one or more colouring materials chosen from soluble dyes and pulverulent colouring materials, such as pigments, pearlescent agents and glitters, well known to a person skilled in the art.

The colouring materials can be present in the composition in a content ranging from 0.01% to 50% by weight, with respect to the weight of the composition, preferably from 0.01% to 30% by weight.

Pigments should be understood as meaning white or coloured and inorganic or organic particles of any shape which are insoluble in the physiological medium and which are intended to colour the composition.

Pearlescent agents should be understood as meaning iridescent particles of any shape, in particular produced by certain molluscs in their shells or else synthesized.

The pigments can be white or coloured and inorganic or organic. Mention may be made, among inorganic pigments, of titanium dioxide, optionally surface treated, zirconium or cerium oxides, zinc, iron or chromium oxides (the iron oxides being black, yellow or red), manganese violet, ultramarine blue, chromium hydrate, ferric blue or metal powders, such as aluminium powder or copper powder.

Mention may be made, among organic pigments, of carbon black, pigments of D & C type, and lakes based on cochineal carmine of barium, strontium, calcium or aluminium.

Mention may also be made of effect pigments, such as particles comprising an organic or inorganic and natural or synthetic substrate, for example glass, acrylic resins, polyester, polyurethane, polyethylene terephthalate, ceramics or aluminas, which may or may not be covered with metal substances, such as aluminium, gold, copper or bronze, or with metal oxides, such as titanium dioxide, iron oxide or chromium oxide, or with inorganic or organic pigments, and their mixtures.

The pearlescent pigments can be chosen from white pearlescent pigments, such as mica covered with titanium oxide or with bismuth oxychloride, coloured pearlescent pigments, such as titanium oxide-coated mica covered with iron oxides, titanium oxide-coated mica covered with in particular ferric blue or with chromium oxide, or titanium oxide-coated mica covered with an organic pigment of the abovementioned type, and pearlescent pigments based on bismuth oxychloride.

Use may also be made of pigments possessing goniochromatic properties, in particular liquid crystal or multilayer pigments.

The dyes are, for example, Sudan red, DC red 17, DC green 6, β-carotene, soybean oil, Sudan brown, DC yellow 11, DC violet 2, DC orange 5 or quinoline yellow.

The colouring material can also be chosen from optical brighteners.

The composition can additionally comprise fibres optionally coated with optical brighteners.

Filler

The composition according to the invention can additionally comprise one or more fillers, in particular in a content ranging from 0.01% to 50% by weight, with respect to the total weight of the composition, preferably ranging from 0.01% to 30% by weight. Fillers should be understood as meaning colourless or white and inorganic or synthetic particles of any shape which are insoluble in the medium of the composition, whatever the temperature at which the composition is manufactured. These fillers are used in particular to modify the rheology or the texture of the composition.

The fillers can be inorganic or organic and of any shape, platelet, spherical or oblong, whatever the crystallographic form (for example sheet, cubic, hexagonal, orthorhombic, and the like). Mention may be made of talc, mica, silica, kaolin, polyamide (Nylon®) powders (Orgasol® from Atochem), poly-β-alanine powders, polyethylene powders, powders formed of tetrafluoroethylene polymers (Teflon®), lauroyl lysine, starch, boron nitride, polymeric hollow microspheres, such as those of polyvinylidene chloride/acrylonitrile, for example Expancel® (Nobel Industrie), or of acrylic acid copolymers (Polytrap® from Dow Corning), silicone resin microbeads (Tospearls® from Toshiba, for example), polyorganosiloxane elastomer particles, precipitated calcium carbonate, magnesium carbonate, basic magnesium carbonate, hydroxyapatite, hollow silica microspheres (Silica Beads® from Maprecos), glass or ceramic microcapsules, or metal soaps derived from organic carboxylic acids having from 8 to 22 carbon atoms, preferably from 12 to 18 carbon atoms, for example zinc stearate, magnesium stearate, lithium stearate, zinc laurate or magnesium myristate.

Additives

The composition can additionally comprise other ingredients commonly used in cosmetic compositions and known to a person skilled in the art as being capable of being incorporated in a nail varnish composition.

Such ingredients can be chosen from oils, waxes, agents for combating free radicals, spreading agents, wetting agents, dispersing agents, antifoaming agents, preservatives, surfactants, fragrances, neutralizing agents, stabilizing agents, antioxidants, active principles which can be chosen from the essential oils, UV screening agents/sunscreens, moisturizing agents, vitamins, proteins, ceramides, plant extracts, and the like, and their mixtures.

The compositions according to the invention can additionally comprise, as active principles, agents for caring for the nails, such as hardening agents for keratinous substances, active principles which act on the growth of the nail, such as, for example, methylsulphonylmethane, and/or active principles for treating various conditions located on the nail, such as, for example, onychomycosis.

Of course, a person skilled in the art will take care to choose this or these optional additional compounds and/or their amounts so that the advantageous properties of the composition for the use according to the invention are not, or not substantially, detrimentally affected by the envisaged addition.

According to another aspect, a subject-matter of the invention is a nail varnish product comprising: i) a container delimiting at least one compartment, the said container being closed by a closing element, and ii) a composition according to the invention which is positioned inside the said compartment.

The container can have any appropriate form. It can in particular be in the form of a bottle and can, at least in part, be made of a material such as glass. However, materials other than glass can be used, such as thermoplastics, for example PP or PE, or such as a metal.

The closing element can be coupled to the compartment by screwing into the closed position of the container. Alternatively, the coupling between the closing element and the container can be carried out other than by screwing, in particular by snapping.

The container is preferably equipped with an applicator which can be in the form of a brush composed of at least one tuft of hairs. Alternatively, the applicator is provided in a form other than a brush, for example in the form of a spatula or of a foam tip.

II—Flexible Article

In general, the article in accordance with the present invention is in the form of a film or laminate.

For the purposes of the present invention, the term “flexible” qualifies a sufficient flexibility of this film, i.e. a flexibility suitable for mechanical deformations of stretching type to adjust it to the surface of a nail.

Moreover, the term “flexible” should also be understood as meaning capable of being non-elastically deformed so as to marry the more or less convex profile of the nail.

This deformability is especially characterized by the deformation at break Cᵣ discussed hereinbelow. The article according to the invention differs especially in this respect from an article of false nail type, which is characterized by a rigidity that is incompatible with such a mechanical deformation.

Another difference between the article in accordance with the invention and a false nail lies in the sensitivity of this article towards polar organic solvents of the type such as acetone, and short esters and/or alcohols, such as alkyl acetates, especially ethyl acetate.

Specifically, the article according to the invention may be readily removed by cleansing using a standard dissolving agent or solvent, as opposed to a false nail, which is taken off.

The invention also relates to an article that may be cleansed of makeup using a solvent chosen from acetone, alkyl acetates such as ethyl acetate, and mixtures thereof.

In all these respects, the flexible article according to the invention differs, on the one hand, from standard liquid compositions such as nail varnishes on account of its partially dry structure, and, on the other hand, from solid products such as false nails due to the fact that, before application, the said article is mechanically deformable and can be cleansed of makeup, unlike a false nail, which is placed directly onto the nail and then filed down to be adjusted, and subsequently directly removed from the nail in order to regain a nail in the normal state.

The article according to the invention may be used for makeup purposes, in which case it comprises dyestuffs, or for purposes of protection with respect to a film of varnish. In this alternative, the polymer layer is generally transparent

A subject of the invention is also a flexible article intended to be applied to the nails and/or false nails to make them up and/or care for them as defined above, also comprising at least one adhesive layer.

The said adhesive layer may also additionally comprise a plasticizer.

In particular, the flexible article may also comprise a protective film in contact with the first face of the adhesive layer, to be removed before placing the article onto the nail. Preferably, the face of the protective film that is in contact with the first face of the foil is covered with a nonstick material, especially a silicone material.

According to one particular embodiment, the article in accordance with the invention is coated on both faces with a removable film, which may be identical or different.

According to one embodiment, the flexible article according to the present invention has a thickness ranging from 6 μm to 1 mm, in particular from 10 μm to 500 μm and even more particularly from 50 μm to 200 μm.

The targeted thickness is understood as being the thickness before application to the nail of the entirety of the indissociable structure containing one or more layers especially comprising the adhesive layer.

In particular, the thickness is understood as being the thickness of all of the polymer and adhesive layers.

On the other hand, any structure removably attached to the flexible article, in particular a protective film on one or other of the faces of the article, in particular a silicone film on the adhesive face of the article, is not taken into account in the measurement of the thickness.

The flexible article, and in particular the excess, may be precut or trimmed, before or after its application, to the desired size and shape using small scissors or nail clippers, or by scratching the film.

The flexible article or flexible article in dry form according to the invention is especially in the form of a non-liquid film, which may be characterized by a high solids content. Specifically, the amount of solids in this article in dry form, i.e. once applied to the nail or the false nail, is greater than or equal to 95% by weight relative to the total weight of the article. In other words, the amount of volatile solvent is less than or equal to 5% by weight relative to the total weight of the article.

According to the second aspect mentioned previously, the makeup product includes an article in partially dry form. In this particular case, the article is packaged in a reservoir, for instance a flexible or inflexible pocket, which is sufficiently leaktight for it to preserve this partially dry appearance. It is not until the time of its use, and consequently of placing it in contact with air, that the product dries out totally to acquire the solids content described previously, in accordance with the article in accordance with the invention.

As mentioned previously, in a product according to the invention, the partially dry article advantageously has a solids content of greater than 80%, especially greater than 85% and less than 95% by weight relative to its total weight. When it is extracted from the packaging of a product in accordance with the invention and exposed to the ambient air, such an article acquires a dry state as defined above after 24 hours.

Preferably, the amount of solids, commonly referred to as the “dry extract”, of the articles according to the invention is measured by heating the sample with infrared rays with a wavelength of from 2 μm to 3.5 μm. The substances contained in said films that have a high vapor pressure evaporate under the effect of this irradiation. Measurement of the weight loss of the sample makes it possible to determine the “dry extract” of the article. These measurements are performed using an LP 16 commercial infrared desiccator from Mettler. This technique is fully described in the machine documentation supplied by Mettler.

The measuring protocol is as follows.

About 10 g of sample of an article are deposited on a metal crucible. After placing it in the desiccator, this crucible is subjected to a nominal temperature of 120° C. for one hour. The wet mass of the sample, corresponding to the initial mass, and the dry mass of the sample, corresponding to the mass after exposure to radiation, are measured using a precision balance.

The solids content is calculated in the following manner:

Dry extract=100×(dry mass/wet mass).

Water Uptake

The article according to the invention may be characterized in dry form by an uptake of water brought to 25° C. of less than or equal to 20%, especially less than or equal to 16% and in particular less than 10%.

According to the present patent application, the teen “water uptake” means the percentage of water absorbed by the article after 60 minutes of immersion in water at 25° C. (room temperature). The water uptake is measured for pieces of about 1 cm² cut from the dry article. They are weighed (mass measurement M2) and then immersed in the water for 60 minutes; after immersion, the piece of film is wiped to remove the excess surface water and then weighed (mass measurement M2). The difference M2−M1 corresponds to the amount of water absorbed by the film.

The water uptake is equal to [(M2−M1)/M1]×100 and is expressed as a weight percentage relative to the weight of the film.

Storage Modulus F

Moreover, the article according to the invention is advantageously a film with a storage modulus of greater than or equal to 1 MPa, especially ranging from 1 MPa to 5000 MPa, in particular greater than or equal to 5 MPa, especially ranging from 5 to 1000 MPa, and more particularly greater than or equal to 10 MPa, for example ranging from 10 to 500 MPa, at a temperature of 30° C. and a frequency of 0.1 Hz.

The storage modulus is measured by DMTA (Dynamic and Mechanical Temperature Analysis).

Viscoelasticimetry tests are performed with a DMTA machine from Polymer TA Instruments (model DMA2980) on a sample of article. The specimens are cut out (for example using a sample punch). These specimens are typically about 150 μm thick, from 5 to 10 mm wide and have a useful length of about 10 to 15 mm.

The measurements are taken at a constant temperature of 30° C.

The sample is subjected to tension and to small bending (for example, a sinusoidal displacement of ±8 μm is imposed thereon) during a frequency scan, the frequency ranging from 0.1 to 20 Hz. The test is thus performed in the linear region, at low levels of bending.

These measurements make it possible to determine the complex modulus E*=E′+iE″ of the test film of composition, E′ being the storage modulus and E″ the loss modulus.

Ultimate Strain and/or Energy at Break

Advantageously, the articles according to the invention have an ultimate strain Eᵣ of greater than or equal to 5%, especially ranging from 5% to 500%, preferably greater than or equal to 15% and especially ranging from 15% to 400%, and/or an energy at break per unit volume Wᵣ of greater than or equal to 0.2 J/cm³, especially ranging from 0.2 to 100 J/cm³, preferably greater than 1 J/cm³ and especially ranging from 1 to 50 J/cm³.

The ultimate strain and the energy at break per unit volume are determined by tensile tests performed on a crosslinked film about 200 μm thick.

To perform these tests, the article is cut into dumbbell-shaped specimens with a useful length of 33±1 mm and a useful width of 6 mm.

The cross section (S) of the specimen is then defined as being:

S=width×thickness (cm²); this cross section will be used for calculating the stress.

The tests are performed, for example, on a tensile testing machine sold under the name Lloyd® LR5K. The measurements are performed at room temperature (20° C.).

The specimens are pulled at a traveling speed of 33 mm/minute, corresponding to a rate of 100% elongation per minute.

A traveling speed is thus imposed and the elongation ΔL of the specimen and the force F required to impose this elongation are simultaneously measured.

From these data ΔL and F, the stress σ strain c parameters are determined. A curve is thus obtained of stress σ=(F/S) as a function of the strain ε=(ΔL/Lₒ)×100, the test being performed up to the breaking point of the specimen, Lₒ being the initial length of the specimen. The ultimate strain εᵣ is the maximum deformation of the sample before the breaking point (in %). The energy at break per unit volume Wᵣ in J/cm³ is defined as the area under this stress/strain curve such that:

$$Wr = {\int }_{0}^{{ɛ}_{r}} σ \cdot ɛ \cdot d ɛ$$

Preferably, the flexible article in accordance with the invention comprises more than 80%, preferably more than 90% and preferably more than 95% by weight of compounds of natural origin relative to the total weight of the flexible article, and preferably the flexible article exclusively comprises compounds of natural origin.

Adhesive Layer

The article according to the invention may have an adhesive outer face if the polymer layer is not self-adhesive. Such an adhesive face is generally obtained by virtue of the presence of at least one adhesive coat, characterized in that this adhesive coat comprises at least one adhesive material.

For the purposes of the present invention, the term “material” means a polymer or a polymeric system that may comprise one or more polymers of different nature. This adhesive material may also contain a plasticizer.

This adhesive material should have a certain tack power defined by its viscoelastic properties.

The viscoelastic properties of a material are conventionally defined by two characteristic values, which are the following:

the elastic modulus, which represents the elastic behaviour of the material for a given frequency and which is conventionally noted as G′,

the viscous modulus, which represents the viscous behaviour of the material for a given frequency and which is conventionally noted as G″.

These magnitudes are especially defined in the “Handbook of Pressure Sensitive Adhesive Technology” 3rd edition, D. Satas, chapter 9, pp. 155 to 157.

Preferably, the adhesive materials that may be used according to the present invention have viscoelastic properties that are measured at a reference temperature of 35° C. and within a certain frequency range.

In the ease of adhesive materials in the form of a solution or dispersion of polymer in a volatile solvent (such as water, a short ester, a short alcohol, acetone, etc.), the viscoelastic properties of this material are measured under conditions in which it has a volatile solvent content of less than 30% and in particular a volatile solvent content of less than 20%.

The elastic modulus of the material is measured in particular at three different frequencies:

at low frequency, i.e. at 2×10⁻² Hz,

at an intermediate frequency, i.e. at 0.2 Hz,

at high frequency, i.e. at 2 Hz,

and the viscous modulus is measured at a frequency of 0.2 Hz.

These measurements make it possible to evaluate the change in the tack powder of the adhesive material over time.

These viscoelastic properties are measured during dynamic tests under low-amplitude sinusoidal stresses (small deformations) performed at 35° C. over a frequency range of from 2×10⁻² to 20 Hz on a “Haake RS50®” rheometer under a torsion/shear stress, for example in cone-plate geometry (for example with a cone angle of 1°).

Advantageously, said adhesive material satisfies the following conditions:

G′(2 Hz, 35° C.)≧10³ Pa, and

G′(35° C.)≦10⁸ Pa, in particular G′(35° C.)≦10⁷ Pa,

G′(2×10⁻² Hz, 35° C.)≦3×10⁵ Pa,

in which:

G′(2 Hz, 35° C.) is the elastic shear modulus of said adhesive material, measured at a frequency of 2 Hz and at a temperature of 35° C.,

G′(35° C.) is the elastic shear modulus of said adhesive material measured at a temperature of 35° C., for any frequency between 2×10⁻² and 2 Hz,

G′(2×10⁻² Hz, 35° C.) is the elastic shear modulus of said adhesive material, measured at a frequency of 2×10⁻² Hz and at a temperature of 35° C.

In one particular form of the invention, the adhesive material also satisfies the following condition:

G″/G′(0.2 Hz, 35° C.)≧0.35

in which:

G″(0.2 Hz, 35° C.) is the viscous shear modulus of said adhesive material, measured at a frequency of 0.2 Hz and at a temperature of 35° C.,

G′(0.2 Hz, 35° C.) is the elastic shear modulus of said adhesive material, measured at a frequency of 0.2 Hz and at a temperature of 35° C.

In one particular form of the invention:

G′(2 Hz, 35° C.)≧5×10³ Pa, and in particular G′(2 Hz, 35° C.)≧10⁴ Pa.

In another particular form of the invention:

G′(2×10⁻² Hz, 35° C.)≦5×10⁴ Pa.

In particular, the adhesive materials according to the invention satisfy the following four conditions:

G′(2 Hz, 35° C.)≧10⁴ Pa, and

G′(35° C.)≦10⁸ Pa, in particular G′(35° C.)≦10⁷ Pa,

G′(2×10⁻² Hz, 35° C.)≦5×10⁴ Pa, and

G″/G′(0.2 Hz, 35° C.)≧0.35.

In the context of the present invention, the adhesive material comprises an ester of caprylic and/or capric acid and/or of caprylyl and/or capryl alcohol as plasticizer and a plant resin as described in paragraph 1—nail varnish composition in liquid or gelled form. It may also comprise an additional adhesive material.

The additional adhesive materials may be chosen from adhesives of “Pressure Sensitive Adhesives” type, for instance those cited in the “Handbook of Pressure Sensitive Adhesive Technology” 3rd edition, D. Satas.

The additional adhesive materials may in particular be polymers chosen from:

polyurethanes,

acrylic polymers,

butyl rubbers, especially chosen from polyisobutylenes,

ethylene-vinyl acetate polymers,

polyamides optionally modified with fatty chains,

adhesive silicones, especially copolymers of silicone resin and of fluid silicone, such as those sold by Dow Corning under the reference Bio-PSA,

and mixtures thereof.

They may in particular be adhesive copolymers derived from the copolymerization of vinyl monomers with polymeric species, for instance those described in patent U.S. Pat. No. 6,136,296. Adhesive copolymers that may also be suitable for the invention are those described in patent U.S. Pat. No. 5,929,173, having a polymer backbone, with a Tg ranging from 0° C. to 45° C., grafted with chains derived from acrylic and/or methacrylic monomers and having, in contrast, a Tg ranging from 50° C. to 200° C.

These additional adhesive materials may also be polymers chosen from block or statistical copolymers comprising at least one monomer or a combination of monomers whose resulting polymer has a glass transition temperature less than room temperature (25° C.), these monomers or combinations of monomers possibly being chosen from butadiene, ethylene, propylene, isoprene and isobutylene, and mixtures thereof. Examples of such materials are block polymers of the type such as styrene-butadiene-styrene, styrene-(ethylene-butylene)-styrene and styrene-isoprene-styrene, for instance those sold under the trade names Kraton® from Shell Chemical Co. or Vector® from the company Dexco Polymers.

The additional adhesive materials are chosen, for example, from polyisobutylenes with a relative molar mass Mv greater than or equal to 10 000 and less than or equal to 150 000. In particular, this relative molar mass is greater than or equal to 18 000 and less than or equal to 150 000.

As commercial products that are particularly suitable for the present invention, mention may be made of polyisobutylenes with respective relative molar masses Mv of 40 000, 55 000 and 85 000 sold under the respective trade names “Oppanol B 10®”, “Oppanol B 12®” and “Oppanol B 15®” by the company BASF, and mixtures thereof.

According to one particular embodiment, the additional adhesive material comprises at least one natural rubber and at least one non-plant tackifying resin.

For the purposes of the invention, the term “natural rubber” means a natural rubber that is obtained directly from plants, or which may optionally have been modified

especially by chemical reaction. Preferably, the natural rubber does not undergo any chemical modification after extraction.

According to one preferred mode of the invention, the natural rubber is obtained after removal of the aqueous phase from a natural latex of polyisoprene, preferably chosen from rubber tree, Parthernium argentatum (guayule), Solidago virgaurea minuta, Taraxacum koksaghyz, Gutta percha, Gutta balata and sapodilla (Manilkara zapota) latexes, and mixtures thereof.

Preferably, the natural latexes are deproteinized, in particular to reduce the risks of allergies.

Non-plant tackifying resins that may be mentioned include aliphatic or aromatic hydrocarbon-based resins, phenolic resins, styrene resins and coumarone-indene resins.

The adhesive layer also comprises a caprylic and/or capric acid ester or an ester of caprylyl and/or capryl alcohol as plasticizer, as described above. The adhesive layer may also comprise additional plasticizers, which are preferably natural or of natural origin.

Additional Plasticizers

The additional plasticizers in accordance with the invention are preferably chosen from optionally modified compounds of natural origin such as oils comprising at least one fatty acid chosen from capric acid, lauric acid, myristic acid, palmitic acid, stearic acid, oleic acid, ricinoleic acid, linoleic acid, linolenic acid, arachidic acid, gadoleic acid, behenic acid, erucic acid, brassidic acid, cetoleic acid, lignoceric acid and nervonic acid, or a mixture thereof.

In particular, the oils that are suitable for use in the invention are chosen from triglycerides formed from fatty acid esters of glycerol in which the fatty acids may have chain lengths ranging from C₄ to C₂₄, these chains possibly being linear or branched, and saturated or unsaturated. These oils are especially heptanoic or octanoic triglycerides, groundnut oil, babassu oils, coconut oil, grape seed oil, cotton seed oil, corn oil, corn germ oil, mustard seed oil, palm oil, rapeseed oil, sesame seed oil, soybean oil, sunflower oil, wheatgerm oil, canola oil, apricot oil, mango oil, castor oil, shea oil, avocado oil, olive oil, sweet almond oil, peach stone oil, walnut oil, hazelnut oil, macadamia oil, jojoba oil, alfalfa oil, poppy oil, pumpkin oil, marrow oil, blackcurrant oil, evening primrose oil, millet oil, barley oil, quina oil, rye oil, sunflower oil, candlenut oil, passion flower oil, musk rose oil or shea butter oil, and mixtures thereof.

According to one particular embodiment, the adhesive layers according to the invention comprise from 0.1% to 70% by weight, especially from 5% to 60% by weight and more particularly from 10% to 50% by weight of plasticizer relative to the total weight of solids of the adhesive layer, it being understood that the plasticizer is formed from the ester of caprylic and/or capric acid or from the ester of caprylyl and/or capryl alcohol and from any other possible additional plasticizer.

According to one particular mode of the invention, the adhesive layer is composed of at least one natural rubber, optionally deproteinized, such as a natural polyisoprene latex freed of its aqueous phase, of at least one plant resin from among those mentioned hereinabove, and of at least one plasticizer as defined above.

Polysaccharides and Polysaccharide Derivatives

The polysaccharides or polysaccharide derivatives are as described above in paragraph I—nail varnish composition in liquid or gelled form.

According to one particular embodiment, the adhesive layer according to the invention comprises from 0 to 60% by weight, especially from 5% to 40% by weight and more particularly from 10% to 30% by weight of polysaccharides or polysaccharide derivatives relative to the total weight of solids of the adhesive layer.

The preferred polysaccharide derivative is ethylguar or ethylcellulose.

Advantageously, the article comprises ethylguar in a content ranging from 10% to 20% by weight relative to the total weight of solids of the adhesive layer.

Preferably, the adhesive layer in accordance with the invention comprises more than 80% by weight, preferably more than 90% by weight and preferably more than 95% by weight of compounds of natural origin relative to the total weight of solids of the adhesive layer.

In particular, the adhesive layer may be formed exclusively from compounds of natural origin.

The adhesive layer in the article in accordance with the invention is generally in the form of a layer having a thickness of 5 to 100 μm, in particular from 10 to 80 μm, preferably from 15 to 50 μm and preferably from 20 to 30 μm.

Polymer Layer

The article according to the present invention may comprise at least one polymer layer, which may comprise at least one natural latex, optionally deproteinized, preferably a natural polyisoprene latex.

Specifically, it has been demonstrated that the use of natural latex and especially of natural polyisoprene latex to form the polymer layer of an adhesive film makes it possible to form films that are both flexible and strong, which can be easily stretched during application to the nails.

In the context of the present invention, the term “natural latex” means a latex of natural origin, i.e. a latex that is obtained directly from plants, and which may optionally have been modified especially by chemical reaction. Preferably, the “natural latex” has not undergone any chemical modification.

According to a particular embodiment, the natural latex is distinct from a natural rubber.

According to one preferred mode of the invention, the natural polyisoprene latex is chosen from rubber tree, Parthernium argentatum (guayule), Solidago virgaurea minuta, Taraxacum koksaghyz, Gutta percha, Gutta balata and sapodilla (Manilkara zapota) latexes, and mixtures thereof.

Preferably, the latexes are deproteinized, in particular to reduce the risks of allergies.

Preferably, the polymer layer comprises a rubber tree latex, or even is formed therefrom.

Rubber tree latexes in accordance with the invention are available from the company SRI Hybrid in the Selatex range, from the company Getahindus in the Loprotex range, or from the company Vystar Corporation in the Vytex range. Guayule latexes in accordance with the invention are available from the company Yulex. Gutta percha latexes are available from the company Rimpex Rubber.

Advantageously, the article comprises natural latex in a content ranging from 30% to 100%, preferably 50% to 100% and more particularly from 70% to 100% by weight of natural latex relative to the total weight of solids of the polymer layer. (The polymer layer may also contain dyestuffs, sunscreens, other film-forming agents, fillers, etc.).

Preferably, the polymer layer in accordance with the invention comprises more than 80%, preferably more than 90% and preferably more than 95% by weight of compounds of natural origin relative to the total weight of the polymer layer, and preferably the polymer layer exclusively comprises compounds of natural origin.

According to one particular embodiment, the flexible article in accordance with the invention comprises a polymer layer, which comprises rubber tree latex, for example deproteinized latex, and an adhesive layer, which comprises sandarac gum, dammar resin, elemi gum, copal resins, benzoin resin or mastic gum, or mixtures thereof, and at least one plasticizer chosen from caprylic/capric acid triglycerides or esters, and optionally ethylguar and a natural latex.

Solvent of Plant Origin

The article according to the invention may comprise a solvent medium formed from one or more solvents of plant origin.

According to one particular embodiment, the flexible article or flexible article in dry form according to the invention comprises from 1% to 20% by weight, especially from 1% to 10% by weight and in particular from 1% to 5% by weight of solvent medium relative to the total weight of the flexible article.

Additional Film-Forming Polymer

The article according to the invention may comprise an additional film-forming polymer as described in the above paragraph, in particular in the same contents.

Auxiliary Film-Forming Agent

The polymer layer may also comprise an auxiliary film-forming agent, which may be chosen from any compound known to those skilled in the art as being capable of satisfying the desired function, and may be chosen especially from plasticizers and coalescers for the film-forming polymer.

In particular, mention may be made of hydrocarbon-based oils such as Parleam and polyisobutenes.

Auxiliary film-forming agents that may also be mentioned include the plasticizers as described above, including the esters of caprylic and/or capric acid or the esters of caprylyl and/or capryl alcohol also described above.

Mention may also be made of common plasticizers or coalescers such as: glycols and derivatives thereof such as diethylene glycol ethyl ether, diethylene glycol methyl ether, diethylene glycol butyl ether, diethylene glycol hexyl ether, ethylene glycol ethyl ether, ethylene glycol butyl ether or ethylene glycol hexyl ether; glycol esters, propylene glycol derivatives and in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether or propylene glycol butyl ether, acid esters, especially carboxylic acid esters, such as citrates, especially triethyl citrate, tributyl citrate, triethyl acetylcitrate, tributyl acetylcitrate or 2-triethylhexyl acetylcitrate; phthalates, especially diethyl phthalate, dibutyl phthalate, dioctyl phthalate, dipentyl phthalate or dimethoxyethyl phthalate; phosphates, especially tricresyl phosphate, tributyl phosphate, triphenyl phosphate or tributoxyethyl phosphate; tartrates, especially dibutyl tartrate; adipates; carbonates; sebacates; benzyl benzoate, butyl acetylricinoleate, glyceryl acetyl-ricinoleate, butyl glycolate, camphor, glyceryl triacetate or N-ethyl-o,p-toluenesulfonamide, oxyethylenated derivatives such as oxyethylenated oils; silicone oils, mixtures thereof.

According to a particular embodiment, the present invention is directed to a flexible article comprising a polymeric layer comprising a natural latex distinct from a natural rubber and an adhesive layer comprising a polysaccharide or a polysaccharide derivative.

The type and amount of plasticizer and/or coalescer may be chosen by a person skilled in the art on the basis of his general knowledge. For example, the plasticizer and/or coalescer content may range from 0.01% to 20% and in particular from 0.5% to 10% by weight relative to the total weight of the polymer layer.

Other Additives

i) Pigments and Dyes

The adhesive layer and the polymer layer of the article according to the invention may especially comprise at least one organic or mineral dyestuff, especially such as the pigments or nacres conventionally used in cosmetic compositions.

The term “pigments” should be understood as meaning white or coloured, mineral or organic particles that are insoluble in an aqueous solution, which are intended to colour and/or opacify the resulting film.

The pigments may be present in a proportion of from 0.01% to 20% by weight, especially from 0.01% to 15% by weight and in particular from 0.02% to 10% by weight relative to the total weight of the first composition and/or of the organic and/or mineral film.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

The dyestuff may also comprise a pigment having a structure that may be, for example, of silica microsphere type containing iron oxide. An example of a pigment having this structure is the product sold by the company Miyoshi under the reference PC Ball PC-LL-100 P, this pigment consisting of silica microspheres containing yellow iron oxide.

Among the organic pigments that may be used in the invention, mention may be made of carbon black, pigments of D & C type, lakes based on cochineal carmine or on barium, strontium, calcium or aluminium, or alternatively the diketopyrrolopyrroles (DPP) described in documents EP-A-542669, EP-A-787730, EP-A-787731 and WO-A-96/08537.

The terms “nacres” should be understood as meaning coloured particles of any form, which may or may not be iridescent, especially produced by certain molluscs in their shell, or alternatively synthesized, and which have a colour effect via optical interference.

The nacres may be chosen from nacreous pigments such as titanium mica coated with an iron oxide, mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the commercially available nacres that may be mentioned are the nacres Timica, Flamenco and Duochrome (on mica base) sold by the company Engelhard, the Timiron nacres sold by the company Merck, the Prestige nacres on mica base sold by the company Eckart and the Sunshine nacres on synthetic mica base sold by the company Sun Chemical.

The nacres may more particularly have a yellow, pink, red, bronze, orange, brown, gold and/or coppery colour or tint.

As illustrations of nacres that may be used in the context of the present invention, mention may be made of gold-coloured nacres sold especially by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company Merck under the names Bronze fine (17384) (Colorona) and Bronze (17353) (Colorona) and by the company Engelhard under the name Super bronze (Cloisonne); the orange nacres sold especially by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold especially by the company Engelhard under the names Nu-antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red-tinted nacres with a golden tint sold especially by the company Engelhard under the name Sunstone G012 (Gemtone); the pink nacres sold especially by the company Engelhard under the name Tan opale G005 (Gemtone); the black nacres with a golden tint sold especially by the company Engelhard under the name Nu antique bronze 240 AB (Timica); the blue nacres sold especially by the company Merck under the name Matte blue (17433) (Microna); the white nacres with a silvery tint sold especially by the company Merck under the name Xirona Silver; and the golden-green pinkish-orange nacres sold especially by the company Merck under the name Indian summer (Xirona), and mixtures thereof.

The first composition and/or the organic and/or mineral film according to the invention may also comprise water-soluble or liposoluble dyes in a content ranging from 0.01% to 10% by weight and especially ranging from 0.01% to 5% by weight relative to the total weight of the first composition or the organic and/or mineral film. The liposoluble dyes are, for example, Sudan Red, DC Red 17, DC Green 6, β-carotene, soybean oil, Sudan Brown, DC Yellow 11, DC Violet 2, DC Orange 5 and quinoline yellow. The water-soluble dyes are, for example, beetroot juice or methylene blue.

ii) Material with an Effect

The adhesive layer and the polymer layer according to the invention may contain at least one material with a specific optical effect. This effect is different from a simple conventional shade effect, i.e. a unified and stabilized effect as produced by the standard dyestuffs described above, for instance monochromatic pigments. For the purposes of the invention, “stabilized” means lacking any effect of variability of the colour according to the angle of observation or in response to a change in temperature.

This material is present in an amount that is sufficient to produce an optical effect that is perceptible to the naked eye. Advantageously, it is an effect chosen from goniochromatic effects, metallic and especially mirror effects, soft-focus effects, rainbow effects and/or thermochromic and/or photochromic effects.

For example, this material may be chosen from particles with a metallic tint, goniochromatic colouring agents, diffractive pigments, thermochromic agents, optical brighteners and fibres, especially interference fibres. Needless to say, these various materials may be combined in order simultaneously to afford two effects, or even a novel effect in accordance with the invention.

Particles with a Metallic Tint

The term “particles with a metallic tint” denotes particles whose nature, size, structure and surface state allow them to reflect the incident light, especially in a non-iridescent manner.

Particles with a substantially flat outer surface are also suitable, since they can, if their size, structure and surface state allow it, more easily give rise to a strong specular reflection, which may then be termed a mirror effect.

The particles with a metallic tint that may be used in the invention may, for example, reflect light in all the components of the visible region without significantly absorbing one or more wavelengths. The spectral reflectance of these particles may, for example, be greater than 70% and better still at least 80%, or even 90% or 95%, in the range 400-700 nm.

These particles generally have a thickness of less than or equal to 1 μm, especially less than or equal to 0.7 μm and in particular less than or equal to 0.5 μm.

The total proportion of particles with a metallic tint is especially less than or equal to 20% by weight and in particular less than or equal to 10% by weight relative to the total weight of the first composition or of the organic and/or mineral film.

The particles with a metallic tint that may be used in the invention are in particular chosen from:

particles of at least one metal and/or of at least one metal derivative,

particles comprising a monomaterial or multimaterial organic or mineral substrate, at least partially coated with at least one layer with a metallic tint comprising at least one metal and/or at least one metal derivative, and

mixtures of the said particles.

Among the metals that may be present in the said particles, mention may be made, for example, of Ag, Au, Cu, Al, Ni, Sn, Mg, Cr, Mo, Ti, Zr, Pt, Va, Rb, W, Zn, Ge, Te and Se and mixtures or alloys thereof. Ag, Au, Cu, Al, Zn, Ni, Mo and Cr, and mixtures or alloys thereof (for example bronzes and brasses) are preferred metals.

The term “metal derivatives” is intended to denote compounds derived from metals, especially oxides, fluorides, chlorides and sulfides.

Among the metal derivatives that may be present in the said particles, mention may be made especially of metal oxides, for instance titanium oxide, especially TiO₂, iron oxide, especially Fe₂O₃, tin oxide, chromium oxide, barium sulfate and the following compounds: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and mixtures or alloys thereof.

According to a first variant, the particles with a metallic tint may be composed of at least one metal as defined above, of at least one metal derivative as defined above, or of a mixture thereof.

These particles may be at least partially covered with a layer of another material, for example of transparent material such as, especially, rosin, silica, stearates, polysiloxanes, polyester resins, epoxy resins, polyurethane resins or acrylic resins.

Illustrations of these particles that may be mentioned include aluminium particles, such as those sold under the names Starbrite 1200 EAC® by the company Siberline and Metalure® by the company Eckart.

Mention may also be made of metal powders of copper or of alloy mixtures such as the references 2844 sold by the company Radium Bronze, metallic pigments, for instance aluminium or bronze, such as those sold under the names Rotosafe® 700 from the company Eckart, silica-coated aluminium particles sold under the name Visionaire Bright Silver® from the company Eckart, and metal alloy particles, for instance the silica-coated bronze (alloy of copper and zinc) powders sold under the name Visionaire Bright Natural Gold® from the company Eckart.

According to a second variant, these particles may be particles comprising a substrate, which thus have a multilayer structure, for example a two-layer structure. This substrate may be organic or mineral, natural or synthetic, monomaterial or multimaterial, solid or hollow. When the substrate is synthetic, it may be made in a form promoting the formation of a reflective surface after coating, especially after depositing a layer of materials with a metallic tint. The substrate may, for example, have a flat surface and the layer of materials with a metallic tint may have a substantially uniform thickness.

The substrate may be chosen in particular from the metals and metal derivatives as mentioned above, and also from glasses, ceramics, aluminas, silicas, silicates and especially aluminosilicates and borosilicates, synthetic mica such as fluorophlogopite, and mixtures thereof, this list not being limiting.

The layer with a metallic tint may totally or partially coat the substrate and this layer may be at least partially covered with a layer of another material, for example a transparent material especially as mentioned above. According to one particular embodiment, this layer with a metallic tint totally coats the substrate directly or indirectly, i.e. with interposition of at least one metallic or non-metallic intermediate layer.

The metals or metal derivatives that may be used in the reflective coat are as defined above. For example, it may be formed from at least one metal chosen from silver, aluminium, chromium, nickel, molybdenum, gold, copper, tin and magnesium, and mixtures (alloys) thereof. Silver, chromium, nickel and molybdenum, and mixtures thereof, are more particularly used.

As illustrations of particles of this second type, mention may be made more particularly of:

Glass particles coated with a metallic layer, especially those described in documents JP-A-09188830, JP-A-10158450, JP-A-10158541, JP-A-07258460 and JP-A-05017710.

As illustrations of these particles comprising a glass substrate, mention may be made of those coated, respectively, with silver, gold or titanium, in the form of platelets, sold by the company Nippon Sheet Glass under the name Microglass Metashine. Particles comprising a silver-coated glass substrate, in the form of platelets, are sold under the name Microglass Metashine REFSX 2025 PS by the company Toyal. Particles comprising a glass substrate coated with nickel/chromium/molybdenum alloy are sold under the name Crystal Star GF 550 and GF 2525 by this same company. Those coated either with brown iron oxide or with titanium oxide, tin oxide or a mixture thereof, for instance those sold under the name Reflecks® by the company Engelhard or those sold under the name Metashine MC 2080GP by the company Nippon Sheet Glass.

These metal-coated glass particles may be coated with silica, for instance those sold under the name Metashine series PSS1 or GPS1 by the company Nippon Sheet Glass.

Particles comprising a spherical glass substrate optionally coated with a metal, especially those sold under the name Prizmalite Microsphere by the company Prizmalite Industries.

Pigments of the Metashine 1080R range sold by the company Nippon Sheet Glass Co. Ltd are also suitable for the invention. These pigments, more particularly described in patent application JP 2001-11340, are C-Glass glass flakes comprising 65% to 72% SiO₂, coated with a layer of titanium oxide of rutile type (TiO₂). These glass flakes have a mean thickness of 1 micron and a mean size of 80 microns, i.e. a mean size/mean thickness ratio of 80. They have blue, green or yellow tints or a silver shade depending on the thickness of the TiO₂ layer.

Particles comprising a silver-coated borosilicate substrate, also known as “white nacres”.

Particles comprising a metal substrate such as aluminium, copper or bronze, in the form of platelets, are sold under the trade name Starbrite by the company Silberline and under the name Visionaire by the company Eckart.

Particles comprising a synthetic mica substrate coated with titanium dioxide, and for example particles with a size of between 80 and 100 μm, comprising a synthetic mica (fluorophlogopite) substrate coated with titanium dioxide representing 12% of the total weight of the particle, sold under the name Prominence by the company Nihon Koken.

The particles with a metallic tint may also be chosen from particles formed from a stack of at least two layers with different refractive indices. These layers may be of polymeric or metallic nature and may especially include at least one polymer layer.

Thus, the particles with a metallic effect may be particles derived from a multilayer polymer film.

The choice of materials intended to constitute the various layers of the multilayer structure is obviously made so as to give the particles thus formed the desired metallic effect.

Such particles are especially described in WO 99/36477, U.S. Pat. No. 6 299 979 and U.S. Pat. No. 6 387 498 and more particularly identified below in the goniochromatic section.

Diffractive Pigments

For the purposes of the present invention, the term “diffractive pigment” denotes a pigment capable of producing a colour variation according to the angle of observation when lit with white light, on account of the presence of a structure that diffracts light.

A diffractive pigment may comprise a diffracting network capable, for example, of diffracting an incident monochromatic light ray in defined directions.

The diffraction network may comprise a periodic unit, especially a line, the distance between two adjacent units being of the same order of magnitude as the wavelength of the incident light.

When the incident light is polychromatic, the diffraction network will separate the various spectral components of the light and produce a rainbow effect.

Reference may appropriately be made regarding the structure of diffractive pigments to the article “Pigments Exhibiting Diffractive Effects” by Alberto Argoitia and Matt Witzman, 2002, Society of Vacuum coaters, 45^{th} Annual Technical Conference Proceedings 2002.

The diffractive pigment may be made with units having different profiles, especially triangular, symmetrical or non-symmetrical, in gaps, of constant or non-constant width, or sinusoidal.

The spatial frequency of the network and the depth of the units will be chosen as a function of the degree of separation of the various orders desired. The frequency may range, for example, between 500 and 3000 lines per mm.

Preferably, the particles of the diffractive pigment each have a flattened form, and are especially in the form of platelets.

The same pigment particle may comprise two crossed, perpendicular or non-perpendicular diffraction networks.

A possible structure for the diffractive pigment may comprise a layer of a reflective material, covered at least on one side with a layer of a dielectric material. The latter material may give the diffractive pigment better rigidity and durability. The dielectric material may thus be chosen, for example, from the following materials: MgF₂, SiO₂, Al₂O₃, AlF₃, CeF₃, LaF₃, NdF₃, SmF₂, BaF₂, CaF₂, LiF and combinations thereof. The reflective material may be chosen, for example, from metals and alloys thereof, and also from non-metallic reflective materials. Among the metals that may be used, mention may be made of Al, Ag, Cu, Au, Pt, Sn, Ti, Pd, Ni, Co, Rd, Nb and Cr, and compounds, combinations or alloys thereof. Such a reflective material may, by itself, constitute the diffractive pigment, which will then be monolayer.

As a variant, the diffractive pigment may comprise a multilayer structure comprising a core of a dielectric material covered with a reflective layer at least on one side, or even totally encapsulating the core. A layer of a dielectric material may also cover the reflective layer(s). The dielectric material used is then preferably mineral, and may be chosen, for example, from metal fluorides, metal oxides, metal sulfides, metal nitrides, and metal carbides, and combinations thereof. The dielectric material may be in crystalline, semi-crystalline or amorphous form. In this configuration, the dielectric material may be chosen, for example, from the following materials: MgF₂, SiO, SiO₂, Al₂O₃, TiO₂, WO, AlN, BN, B₄C, WC, TiC, TiN, N₄Si₃, ZnS, glass particles and carbons of diamond type, and combinations thereof.

The diffractive pigment used may be chosen especially from those described in the American patent application US 2003/0 031 870 published on 13 Feb. 2003.

A diffractive pigment may comprise, for example, the following structure: MgF₂/Al/MgF₂, a diffractive pigment having this structure being sold under the name Spectraflair 1400 Pigment Silver by the company Flex Products, or Spectraflair 1400 Pigment Silver FG. The weight proportion of MgF₂ may be between 80% and 95% of the total weight of the pigment.

Goniochromatic Colouring Agents

For the purposes of the invention, a goniochromatic colouring agent allows a colour change, also known as a “colour flop”, to be observed as a function of the angle of observation, greater than that which may be encountered with nacres. One or more goniochromatic colouring agents may be used simultaneously.

The goniochromatic colouring agent may be chosen so as to present a relatively large colour change with the angle of observation.

The goniochromatic colouring agent may thus be chosen such that a colour difference ΔE of the cosmetic composition, measured in the CIE 1976 colorimetric space, of at least 2 may be observed for a variation of the angle of observation of between 0° and 80° under illumination at 45°.

The goniochromatic colouring agent may also be chosen such that a variation Dh of the hue angle of the cosmetic composition, in the CIE 1976 plane, of at least 30° or even at least 40° or at least 60°, or even at least 100°, may he observed for an illumination at 45° and a variation of the angle of observation of between 0° and 80°.

The goniochromatic colouring agent may be chosen, for example, from multilayer interference structures and liquid-crystal colouring agents.

In the case of a multilayer structure, it may comprise, for example, at least two layers, each layer, which may or may not be independent of the other layer(s), being made, for example, from at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiOH₂, Ag, Al, Au, Cu, Rh, Ti, Ta, W, Zn, MoS₂, cryolite, and alloys, polymers and combinations thereof.

The multilayer structure may or may not have, relative to a central layer, symmetry in the chemical nature of the stacked layers.

Examples of symmetrical multilayer interference structures that may he used in the compositions prepared in accordance with the invention are, for example, the following structures: Al/SiO₂/Al/SiO₂/Al, pigments having this structure being sold by the company Dupont de Nemours; Cr/MgF₂/Al/MgF₂/Cr, pigments having this structure being sold under the name Chromaflair by the company Flex; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃; pigments having these structures being sold under the name Sicopearl by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona by the company Merck (Darmstadt). By way of example, these pigments may he pigments of silica/titanium oxide/tin oxide structure sold under the name Xirona Magic by the company Merck, pigments of silica/brown iron oxide structure sold under the name Xirona Indian Summer by the company Merck, and pigments of silica/titanium oxide/mica/tin oxide structure sold under the name Xirona Caribbean Blue by the company Merck. Mention may also be made of the Infinite Colors pigments from the company Shiseido. Depending on the thickness and the nature of the various layers, different effects are obtained. Thus, with the Fe₂O₃/SiO₂/Al/SiO₂/Fe₂O₃ structure, the colour changes from green-golden to red-grey for SiO₂ layers of 320 to 350 nm; from red to golden for SiO₂ layers of 380 to 400 nm; from violet to green for SiO₂ layers of 410 to 420 nm; from copper to red for SiO₂ layers of 430 to 440 nm.

Goniochromatic colouring agents with a multilayer structure comprising an alternation of polymeric layers may also be used.

As illustrations of the materials that can constitute the various layers of the multilayer structure, it is possible to mention, this list not being limiting: polyethylene naphthalate (PEN) and its isomers, for example 2,6-, 1,4-, 1,5-, 2,7- and 2,3-PEN, polyalkylene terephthalates, polyimides, polyetherimides, atactic polystyrenes, polycarbonates, polyalkyl methacrylates and polyalkyl acrylates, syndiotactic polystyrene (sPS), syndiotactic poly-alpha-methylstyrenes, syndiotactic polydichlorostyrene, copolymers and blends of these polystyrenes, cellulose derivatives, polyalkylene polymers, fluoropolymers, chloropolymers, polysulfones, polyethersulfones, polyacrylonitriles, polyamides, silicone resins, epoxy resins, polyvinyl acetate, polyetheramides, ionomeric resins, elastomers and polyurethanes. Copolymers are also suitable, for example copolymers of PEN (for example copolymers of 2,6-, 1,4-, 1,5-, 2,7-, and/or 2,3-naphthalenedicarboxylic acid or the esters thereof with (a) terephthalic acid or its esters; (b) isophthalic acid or its esters; (c) phthalic acid or its esters; (d) alkane glycols; (e) cycloalkane glycols (for example cyclohexanedimethanol dial); (f) alkanedicarboxylic acids; and/or (g) cycloalkanedicarboxylic acids, polyalkylene terephthalate copolymers and styrene copolymers. In addition, each individual layer may include blends of two or more of the above polymers or copolymers. The choice of materials intended to constitute the various layers of the multilayer structure is, of course, made so as to give the particles thus formed the desired reflective appearance.

As examples of pigments with a polymeric multilayer structure, mention may be made of those sold by the company 3M under the name Color Glitter.

The liquid-crystal colouring agents comprise, for example, silicones or cellulose ethers onto which are grafted mesomorphic groups.

Examples of liquid-crystal goniochromatic particles that may be used include, for example, those sold by the company Chenix and also the products sold under the name Helicone® HC by the company Wacker.

These agents may also be in the form of dispersed goniochromatic fibres. Such fibres may, for example, have a size of between 50 μm and 700 μm, for example about 300 μm. Interference fibres with a multilayer structure may be used in particular. Fibres with a multilayer structure of polymers are described especially in documents EP-A-0 921 217, EP-A-0 686 858 and U.S. Pat. No. 5,472,798. The multilayer structure may comprise at least two layers, each layer, which may or may not be independent of the other layer(s), being made of at least one synthetic polymer. The polymers present in the fibres may have a refractive index ranging from 1.30 to 1.82 and better still ranging from 1.35 to 1.75. The polymers that are preferred for making the fibres are polyesters such as polyethylene terephthalate, polyethylene naphthalate, polycarbonate; acrylic polymers such as polymethyl methacrylate; polyamides.

Goniochromatic fibres with a polyethylene terephthalate/nylon-6 two-layer structure are sold by the company Teijin under the name Morphotex.

In one variant, this goniochromatic colouring agent may be combined with at least one diffractive pigment.

The combination of these two materials results in a composition or a film that has increased colour variability, and thus which is capable of allowing an observer to see a colour change, or even a colour movement, under various observation and lighting conditions.

The weight ratio of the diffractive pigment relative to the goniochromatic colouring agent is preferably between 85/15 and 15/85, better still between 80/20 and 20/80 and better still between 60/40 and 40/60, for example about 50/50. Such a ratio is favourable towards obtaining a strong rainbow effect and a strong goniochromatic effect.

Optical Brighteners

Optical brighteners are compounds that are well known to those skilled in the art. Such compounds are in particular described in “Fluorescent Whitening Agent, Encyclopedia of Chemical Technology, Kirk-Othmer”, Vol. 11, pp. 227-241, 4th Edition, 1994, Wiley. They may be defined more particularly as compounds that absorb essentially in the UVA range between 300 and 390 nm and re-emit essentially between 400 and 525 nm. Among the optical brighteners that may be mentioned more particularly are stilbene derivatives, in particular polystyrylstilbenes and triazinestilbenes, coumarin derivatives, in particular hydroxycoumarins and aminocoumarins, oxazole, benzoxazole, imidazole, triazole and pyrazoline derivatives, pyrene derivatives and porphyrin derivatives, and mixtures thereof. Such compounds are widely commercially available.

Mention may be made, for example, of: the naphthotriazole stilbene derivative sold under the trade name Tinopal GS, disodium 4,4′-distyrylbiphenylsulfonate (CTFA name: disodium distyrylbiphenyl disulfonate) sold under the trade name Tinopal CBS-X, the cationic aminocoumarin derivative sold under the trade name Tinopal SWN Conc., the sodium 4,4′-bis[(4,6-dianilino-1,3,5-triazin-2-yl)amino]stilbene-2,2′-disulfonate sold under the trade name Tinopal SOP, the 4,4′-bis[(4-anilino-6-bis(2-hydroxyethyl)amino-1,3,5-triazin-2-yl)amino]stilbene-2,2′-disulfonic acid sold under the trade name Tinopal UNPA-GX, the 4,4′-bis[anilino-6-morpholine-1,3,5-triazin-2-yl)amino]stilbene sold under the trade name Tinopal AMS-GX, the disodium 4,4′-bis[(4-anilino-6-(2-hydroxyethyl)methylamino-1,3,5-triazin-2-yl)amino] stilbene-2,2′-sulfonate sold under the trade name Tinopal 5BM-GX, all by the company Ciba Spécialités Chimiques, 2,5-thiophenediylbis(5-tert-butyl-1,3-benzoxazole) sold under the trade name Uvitex OB by the company Ciba, the anionic derivative of diaminostilbene as a dispersion in water, sold under the trade name Leucophor BSB Liquid by the company Clariant, and the optical brightener lakes sold under the trade name “Covazur” by the company Wackherr.

The optical brighteners that may be used in the present invention may also be in the form of copolymers, for example acrylates and/or methacrylates, grafted with optical brightener groups as described in patent application FR 99/10942.

They may be used in unmodified form or introduced into the film in the form of particles and/or fibres coated with the said brightener, such as those described below,

In particular, the fibres coated with optical brightener as sold by the company LCW under the trade reference Fiberlon 54 ZO₃, with a length of about 0.4 mm and a yarn count of 0.5 denier, may be used.

Material with a Relief Effect

The relief effect may or may not be associated with an optical effect. A material of this type is generally present in an amount sufficient to give a relief effect that is perceptible to the touch or even to the naked eye. It may especially be a coarse and/or hammered effect.

Material Imparting a Coarse Appearance

Particles of substantially spherical or ovoid shape may give makeup a soft feel. Advantageously, the solid particles have a substantially spherical shape, to allow good distribution during their application.

The solid particles used according to the invention may have a mean size ranging from 2.5 μm to 5 mm and better still from 50 μm to 2 mm. The smaller the sizes of the particles, the more satisfactory their staying power. The use of particles is also compatible with the production of patterns.

The solid particles may be made of any material that satisfies the density properties defined previously. For example, the solid particles may be made of a material chosen from glass, zirconium oxide, tungsten carbide, plastics such as polyurethanes, polyamides, polytetrafluoroethylene or polypropylene, metals such as steel, copper, brass or chromium, marble, onyx, jade, natural mother-of-pearl, precious stones (diamond, emerald, ruby or sapphire), amethyst or aquamarine. Glass beads are preferably used, such as those sold under the name Silibeads by the company Sigmund Lindner; these beads also have the advantage of equally giving a glossy and glittery effect to makeup.

The solid particles, which may or may not be deformable, may be solid or hollow, colourless or coloured, and coated or uncoated.

As regards the fibres that may be used according to the invention, they may be mineral or organic fibres, of natural or synthetic origin.

The term “fibre” should be understood as meaning an object of length L and diameter D such that L is very much greater than D, D being the diameter of the circle in which the cross section of the fibre is inscribed. In particular, the ratio L/D (or shape factor) is chosen in the range from 3.5 to 2500, preferably from 5 to 500 and better still from 5 to 150.

They may especially be fibres used in the manufacture of textiles, and especially silk fibre, cotton fibre, wool fibre, flax fibre, cellulose fibre extracted in particular from wood, from plants or from algae, rayon fibre, polyamide (^{Nylon}®) fibre, viscose fibre, acetate fibre, especially rayon acetate fibre, poly(p-phenyleneterephthalamide) (or aramid) fibre, especially Kevlar® fibre, acrylic polymer fibre, especially polymethyl methacrylate fibre or poly(2-hydroxyethyl methacrylate)fibre, polyolefin fibre and especially polyethylene or polypropylene fibre, glass fibre, silica fibre, carbon fibre, especially of carbon in graphite form, polytetrafluoroethylene (such as Teflon®) fibre, insoluble collagen fibre, polyester fibre, polyvinyl chloride fibre or polyvinylidene chloride fibre, polyvinyl alcohol fibre, polyacrylonitrile fibre, chitosan fibre, polyurethane fibre, polyethylene phthalate fibre, and fibres formed from a mixture of polymers such as those mentioned above, for instance polyamide/polyester fibres.

Material Imparting a Hammered Appearance

The inventors have also found that it is possible to condition in the invention a material comprising a mixture of fumed silica, metallic pigments and organopolysiloxane compound to give it a hammered appearance.

Such a mixture is especially described in patent application EP 1 040 813.

iii) Material with an Olfactory Effect

Advantageously, the article according to the invention may also have olfactory properties by incorporation, especially into at least one of the layers, of at least one odorous material or fragrancing substance.

The fragrancing substance may be chosen from any odoriferous substance well known to those skilled in the art, and especially from essential oils and/or essences.

This olfactory material may, if necessary, be introduced via a plasticizing solvent.

The term “plasticizing solvent” means a compound that at least partially dissolves the olfactory material and that is capable of evaporating slowly.

The plasticizing solvent may be chosen from glycols such as dipropylene glycol, ethyldiglycol, n-propyl glycol, n-butyl glycol, methyldiglycol, n-butyldiglycol, alcohols such as cyclohexanol, 2-ethylbutanol, 3-methoxybutanol, 2-ethylhexanol, phenoxyethanol, esters such as glycol monoacetate, ethyl glycol acetate, n-butyl glycol acetate, ethyldiglycol acetate, n-butyldiglycol acetate, ethyl abietate, isopropyl myristate, propylene glycol diacetate, propylene glycol methyl ether acetate, glycol ethers such as dipropylene glycol methyl ether and dipropylene glycol butyl ether, alone or as a mixture.

Any one of the layers of the article according to the invention may also contain one or more formulation additives commonly used in cosmetics and more especially in the cosmetics and/or nailcare field. They may be chosen especially from fragrances, vitamins, trace elements, softeners, sequestrants, acidifying or basifying agents, wetting agents, thickeners, dispersants, antifoams, spreading agents, co-resins, preserving agents, UV-screening agents, active agents, moisturizers, neutralizers, stabilizers and antioxidants, and mixtures thereof.

Thus, it may especially incorporate, as active agents, keratin material hardeners or strengtheners, active agents for promoting nail growth, such as methylsulfonylmethane, and/or active agents for treating various nail complaints, for instance antimycotic active agents or antimicrobial agents.

The amounts of these various ingredients are those conventionally used in this field, for example from 0.01% to 20% by weight and especially from 0.01% to 10% by weight relative to the total weight of the flexible film and/or the adhesive material.

The invention is illustrated in greater detail in the example that follows, which is presented as a non-limiting illustration of the invention.

Unless otherwise indicated, the amounts are given as weight percentages of starting material relative to the total weight of the composition.

The invention is illustrated in more detail in the following examples, which are presented by way of illustration and without implied limitation of the invention.

Unless otherwise indicated, the amounts are given as percentages by weight, with respect to the total weight of the composition.

### EXAMPLE 1

| | |
|---|---|
| Cellulose acetate butyrate⁽¹⁾ | 10.0% |
| Benzoin gum⁽²⁾ | 15.0% |
| Ethanol⁽³⁾ | 37.5% |
| Ethyl acetate⁽⁴⁾ | 37.5% |

⁽¹⁾CAB 381 0.5, sold by Eastman Chemical
⁽²⁾sold by WoodFinishing Enterprises
⁽³⁾sold by Tereos
⁽⁴⁾sold by Rhodia

### EXAMPLE 2

| | |
|---|---|
| Cellulose acetate butyrate⁽¹⁾ | 4.2% |
| Ethanol⁽³⁾ | 46.2% |
| Ethyl acetate⁽⁴⁾ | 25.8% |
| Sandarac resin (North African)⁽⁵⁾ | 19.8% |
| Triglycerides of caprylic/capric acids⁽⁶⁾ | 4.0% |

⁽⁵⁾sold by WoodFinishing Enterprises
⁽⁶⁾sold by Cognis under the reference Myritol 318

### EXAMPLE 3

| | |
|---|---|
| Cellulose acetate butyrate⁽¹⁾ | 7.5% |
| Benzoin gum⁽²⁾ | 10.0% |
| Ethanol⁽³⁾ | 25.0% |
| Ethyl acetate⁽⁴⁾ | 47.5% |
| Sandarac resin (North African)⁽⁵⁾ | 10.0% |

### EXAMPLE 4

| | |
|---|---|
| Ethanol⁽³⁾ | 19.5% |
| Ethyl acetate⁽⁴⁾ | 56.0% |
| Ethyl guar⁽⁷⁾ | 6.5% |
| Crushed purified distilled elemi gum⁽⁸⁾ | 15.5% |
| Red iron oxide⁽⁹⁾ | 2.5% |

⁽⁷⁾N-Hance AG 200, sold by Hercules
⁽⁸⁾sold by Emiga
⁽⁹⁾sold by LCW under the reference Natpure Red

### EXAMPLE 5

| | |
|---|---|
| Triglycerides of caprylic/capric acids⁽⁶⁾ | 4.0% |
| Ethanol of plant origin⁽³⁾ | 45.7% |
| Ethyl acetate of plant origin⁽⁴⁾ | 25.3% |
| Cellulose acetate butyrate⁽¹⁾ | 4.2% |
| Sandarac resin (North African)⁽⁵⁾ | 19.8% |
| Pigments | 1.0% |

In order to obtain the compositions of the examples, the ingredients are mixed and then stirred until all the compounds (apart from the pigments) have dissolved and until a good dispersion of the pigments (for the formulations comprising pigments) has been obtained.

After drying, the compositions of Examples 1 to 5 form hard, glossy and adherent films. The compounds in the context of the invention do not prove to be harmful to the additional qualities expected for a cosmetic composition.

### EXAMPLE 6

In this example, a first polymer film is prepared from a deproteinized rubber tree latex⁽⁹⁾ with a solids content of about 60%. The rubber tree latex is coated to a wet thickness of 150 μm onto a contrast card, sold by the company Byk Gardner under the product name Prüfkarte 2853.

A second composition (see the table below) is coated to a wet thickness of 300 μm onto a counteradhesive (Scotchpak 1022 Release linear 3.0 mil available from 3M).

| | |
|---|---|
| Ethylguar⁽⁶⁾ | 5% |
| Elemi gum⁽⁷⁾ | 15% |
| Caprylic/capric acid triglycerides⁽⁶⁾ | 10% |
| Ethyl acetate⁽⁴⁾ | 70% |

⁽⁴⁾sold by the company SRI Hybrid

After drying the two films, the latex film is placed in contact with the film of the composition of the table. A pressure is exerted to make the films adhere together. Next, the contrast card is cautiously removed, followed by the counteradhesive. A adhesive laminate that is strong and flexible is obtained, enabling the adhesive laminate to be easily removed during application to the nails,

1. Nail varnish comprising a solvent medium composed of one or more solvents of plant origin, at least one polysaccharide ester or alkyl ether and at least one plant resin, wherein the solvent medium is chosen from a mixture of ethanol and ethyl acetate.

2. Nail varnish comprising a solvent medium composed of one or more solvents of plant origin, at least one polysaccharide ester or alkyl ether and at least one plant resin, wherein the plant resin is chosen from rosin and its derivatives, terpene resins, gum sandarac, dammars, elemi, copals, benzoin, mastic gum and their mixtures.

3. Nail varnish according to claim 2, wherein the solvent medium of plant origin is chosen from ethanol, and/or acetates.

4. Nail varnish according to claim 2, wherein the solvent medium of plant origin is chosen from ethyl acetate and/or lactates.

5. Nail varnish according to claim 2, wherein the solvent medium is chosen from a mixture of ethanol and ethyl acetate.

6. Nail varnish according to claim 2, wherein it comprises less than 5% by weight of water with respect to the total weight of the nail varnish, indeed is even completely devoid thereof.

7. Nail varnish according to claim 2, wherein the solvent medium is present in a content of between 40 and 90% by weight with respect to the total weight of the nail varnish.

8. Nail varnish according to claim 2, wherein the polysaccharide ester or alkyl ether is chosen from cellulose acetate butyrates, cellulose acetate propionates, ethyl celluloses and ethyl guars.

9. Nail varnish according to claim 2, wherein the polysaccharide ester or alkyl ether is present in a content of between 2 and 30% by weight with respect to the total weight of the nail varnish.

10. Nail varnish according to claim 1, wherein the plant resin is chosen from rosin and its derivatives, terpene resins, gum sandarac, dammars, elemi, copals, benzoin, mastic gum and their mixtures.

11. Nail varnish according to claim 2, wherein the plant resin is present in a content of between 10 and 50% by weight with respect to the total weight of the nail varnish.

12. Nail varnish according to claim 2, wherein it additionally comprises a caprylic/capric alcohol and/or acid ester.

13. Nail varnish according to claim 1, wherein it additionally comprises caprylic and/or capric acid triglyceride.

14. Nail varnish according to claim 12, wherein the caprylic/capric alcohol and/or acid ester is present in a content of between 0.5 and 15% by weight with respect to the total weight of the composition.

15. Nail varnish according to claim 2, wherein it comprises less than 5% by weight of nitrocellulose dry matter, with respect to the total weight of the nail varnish.

16. Nail varnish according to claim 2, wherein it comprises at least one colouring material in a content ranging from 0.01% to 50% by weight, with respect to the weight of the nail varnish.

17. Cosmetic method for making up or for the non-therapeutic care of the nails comprising the application, to the nails, of at least one layer of a nail varnish according to claim 1.

18. Cosmetic method for making up or for the non-therapeutic care of the nails comprising the application, to the nails, of at least one layer of a nail varnish according to claim 2.

19. Flexible article comprising in an adhesive layer, at least one plant resin, at least one plasticizer and at least one solvent of plant origin, wherein the plasticizer is chosen from caprylic and/or capric acid esters or an ester of caprylyl and/or capryl.

20. Article according to the preceding claim, wherein the plant resin is chosen from rosin and its derivatives, terpene resins, gum sandarac, dammars, elemi, copals, benzoin, mastic gum and their mixtures.

21. Article according to claim 19, wherein the solvent medium of plant origin is chosen from ethanol, and/or acetates.

22. Article according to claim 19, wherein the plasticizer is present in a content of between 5% to 60% by weight of plasticizer relative to the total weight of solids of the adhesive layer.

23. Article according to claim 19, wherein the adhesive layer also comprises a polysaccharide or polysaccharide derivative chosen from cellulose or guar alkyl ethers and esters, in a content ranging from 5% to 40% by weight of polysaccharide or polysaccharide derivative relative to the total weight of solids of the adhesive layer.

24. Article according to claim 19, wherein the adhesive layer also comprises ethylguar and optionally a latex of natural origin.

25. Article according to claim 19, comprising at least one polymer layer, wherein the said polymer layer also comprises a latex of natural origin, optionally deproteinized, chosen from rubber tree, Parthernium argentatum, Solidago virgaurea minuta, Taraxacum koksaghyz, Gutta percha, Gutta balata and sapodilla latexes, and mixtures thereof in a content ranging from 30% to 100% by weight of natural latex relative to the total weight of solids of the polymer layer.

26. Article according to claim 19, wherein the said article can be removed by cleansing using a solvent chosen from acetone and alkyl acetates such as ethyl acetate, and mixtures thereof.

27. Article according to claim 19, wherein the weight of solids of the said article is greater than or equal to 95% by weight relative to the total weight of the article.

28. Product for making up and/or caring for the nails and/or false nails, comprising, in substantially airtight packaging, at least one article according to claim 19, the packaging being such that the article therein is kept in a partially dry form.

29. Process for making up and/or caring for the nails and/or false nails, comprising at least one step that consists in applying to a natural nail and/or a false nail an article as defined in claim 19 in dried or partially dry form.
